(19) United States
(12) Patent Application Publication
(10) Pub. No.: US 2019/0380964 A1
(43) Pub. Date: Dec. 19, 2019

(54) METHODS AND COMPOSITIONS FOR MAKING AND USING NANOTHERAPEUTIC DRUG DELIVERY VEHICLES

(71) Applicant: GEORGE MASON UNIVERSITY, Fairfax, VA (US)

(72) Inventors: Nitin Agrawal, Fairfax, VA (US); Steven Andrew Roberts, Fairfax, VA (US)

(21) Appl. No.: 16/444,360

(22) Filed: Jun. 18, 2019

Related U.S. Application Data
(60) Provisional application No. 62/686,920, filed on Jun. 19, 2018.

(51) Int. Cl. A61K 9/127; A61K 9/51

(52) U.S. Cl. CPC A61K 9/1277; B82Y 5/00; A61K 9/5123

(57) ABSTRACT

Disclosed herein are method and compositions for making and using liposomes having small diameters and low polydispersity.

### CROSS REFERENCE TO RELATED APPLICATIONS

This application claims the benefit of and priority under 35 U.S.C. §§ 119, 120 to: U.S. Provisional Patent Application No. 62/686,920 filed Jun. 19, 2018, entitled, “METHODS AND COMPOSITIONS FOR MAKING AND USING NANOTHERAPEUTIC DRUG DELIVERY VEHICLES”, which is incorporated herein by reference in its entirety.

### STATEMENT REGARDING FEDERALLY SPONSORED RESEARCH OR DEVELOPMENT

This invention was made with government support under No. 1645195 and 1550976 awarded by the National Science Foundation. The government has certain rights in the invention.

### FIELD OF THE DISCLOSURE

Disclosed herein are methods for making and liposomal therapeutic delivery compositions that are generally uniform and provide high drug loading capacity.

### BACKGROUND

Liposomes are one of the most studied nano-delivery systems. However, only a handful of formulations have received FDA approval. Existing liposome synthesis techniques are complex and specialized, posing a major impediment in the design and implementation of liposome delivery systems, both as point of care and mass produced therapeutics.

In addition to the synthesis complexities of liposomes, encapsulation of drugs or other active agents at pharmaceutically effective concentrations within liposomes is a major challenge with existing techniques.

Since their initial discovery, liposomes have been recognized for their potential as a drug delivery system (DDS). These colloidal, vesicle-like particles are amphipathic and allow for compartmentalization of both hydrophilic and hydrophobic molecules. By altering their size, affinity, and surface chemistry, the rates of biodegradation, bioavailability, and release of therapeutics at target tissues can be customized. Despite their well-documented use as the delivery vehicles for chemotherapeutics (e.g. Doxil), antibiotics (e.g., Abelcet), and opioids (e.g., Diprivan), only a small number of liposomal drugs have been approved by the U.S. Food and Drug Administratin (FDA). Clearly there is a separation between the development of liposomal delivery systems and their widespread implementation as both theranostic and therapeutic platforms.

A major roadblock in the engineering of potent and therapeutic grade drug delivery systems revolves around complement system activation and scalability of the synthesis procedure. It is widely accepted that small unilamellar liposomes (SULs) with a net neutral charge are the least reactogenic drug delivery systems (DDS's). However, obtaining a population of SULs with low polydispersity index (PDI) and optimal size requires specialized equipment and multiple secondary processing techniques. Thin film deposition is the most common method of producing SULs. Using this approach, larger multilamellar vesicles are synthesized and then the size is altered through separate post-synthesis processing, such as sonication, extrusion, or homogenization. While this method is able to effectively decrease the heterogeneity and number of lamellae, it damages fragile encapsulated molecules, requires tedious and time-consuming protocols (e.g. chromatography or dialysis), and necessitates use of equipment not readily available in laboratory settings (e.g. ultracentrifuge and rotary evaporators). Thus, this approach is very difficult to scale up for the mass production that is necessary for widespread use, and is not applicable for point-of-care applications.

A more simplistic approach towards producing SULs uses a fine tip needle to inject an alcoholic lipid solution into an aqueous medium, such as ultrapure water or saline containing the therapeutic. This method has been shown to reproducibly form SUL's with a narrow PDI, and has been recently implemented on a large scale to mass produce SUL's with a PDI of less than 0.2. A noted drawback of this method is the low encapsulation efficiency of hydrophilic drug molecules because of the high surface area to volume ratio. Thus, a secondary post-processing step to remove excess non-encapsulated molecules and concentrate the solution is necessary. Typically, this is carried out through precipitation methods similar to those previously mentioned (i.e. ultracentrifugation and dialysis), neither of which are readily available in common laboratory settings. Furthermore, precipitation via centrifugation has recently been shown to offer poor recovery due to the low density and sedimentation coefficient associated with nanoscale liposomes, substantially decreasing the overall yield. More recently, several microfluidic approaches to synthesize liposomes have also been demonstrated that provide greater control over mixing of lipid and aqueous solutions. These techniques also require specialized microfabrication facilities and only offer proof-of-concept strategies to investigate lipid self-assembly at small scales.

Thus there exists a need for liposome synthesis techniques that are: 1) rapid; 2) economically suitable for synthesis at scale; and 3) capable of producing concentrated, active agent-encapsulating liposome solutions with low polydispersities and small liposome diameters. The present disclosure provides methods and compositions that overcome these noted difficulties.

### BRIEF SUMMARY OF THE DISCLOSURE

Disclosed herein are methods for synthesis and purification of injectable nanocarriers (SPIN) for rapid and efficient production of small drug-loaded liposomes, which may be accomplished using common benchtop equipment. In at least one embodiment, the present disclosure provides liposome synthesis methods that are, in comparison to previous methods, rapid, economically suitable for synthesis at scale and capable of producing concentrated, active agent-encapsulating liposome solutions that present low polydispersities and small liposomes diameters (<200 nm). In various embodiments, the present methods may provide for solutions including unilamellar liposomes, with mean a diameter of 80 nm and a polydispersity of 0.13. In at least one embodiment, the present methods provide techniques for liposome synthesis that do not require any secondary post-processing techniques. In various embodiments, the presents methods may provide liposomes that encapsulate active agents (for example, dextrans [300-20,000 Da]), representing small and large molecular drug formulations, without significantly affecting one or more characteristics of the liposomes. In one or more embodiments, the present methods provide techniques for removing non-encapsulated molecules from liposomal solutions by using a filter centrifugation step. In at least one embodiment, filter centrifugation may largely eliminate necessities for tedious ultracentrifugation protocols (e.g., to concentrate and purify liposomal solutions). In one or more embodiments, functional efficacy of loaded liposomes as drug delivery vehicles may be validated by encapsulating a fluorescent cell tracker (CMFDA) and observing the liposomal release and subsequent uptake of dye by metastatic breast cancer cells (MDA-MB-231) in vitro. Thus, the disclosed systems, methods and compositions address existing challenges associated with liposome preparation in resource-limited settings and offer significant potential for advances in translational pharmaceutical development.

Briefly described, in one embodiment, the present methods for liposome synthesis may include, but are not limited to: 1) mixing an isopropyl alcohol at a concentration between 5-10%, at least one lipid component and cholesterol to create a first solution, wherein the at least one lipid component and the cholesterol form small unilamellar liposomes within the first solution, wherein mixing further includes: A) vortexing the isopropyl alcohol, at a rate between about 400-800 revolutions per minute, immediately before and throughout adding the at least one lipid component and the cholesterol; and B) vortexing the first solution at a rate between about 400-800 revolutions per minute; 2) heating the first solution to a first temperature between about 45-65 degrees Celsius; 3) concentrating the first solution by performing centrifugal filtration, wherein: A) the centrifugal filtration is performed at a rate of about 6000 g; B) the centrifugal filtration is performed using 100 kDa filtration tubes; and C) the centrifugal filtration is repeated between about 5-10 times; 4) mixing a second solution comprising: A) an aqueous component; and B) an active agent; 5) heating the second solution to the first temperature; 6) injecting the first solution into the second solution at a constant rate of about 400 μL/min to form a mixture, wherein injecting further comprises: A) vortexing, at a predetermined rate between about 400-800 revolutions per minute, the second solution immediately before and throughout injecting the first solution into the second solution; and B) vortexing, at the predetermined rate, the mixture that is formed after injecting the first solution; 7) incubating the mixture at the first temperature for about 1-2 hours, wherein incubating the mixture causes encapsulation of the active agent by the small unilamellar liposomes; 8) cooling the mixture to between about 0-5 degrees Celsius, wherein the cooled mixture comprises active agent-encapsulating small unilamellar liposomes; and 9) concentrating the mixture by performing centrifugal filtration, wherein concentrating the mixture removes a substantial portion of non-encapsulated active agent from the mixture, wherein: A) the centrifugal filtration is performed at a rate of about 6000 g; B) the centrifugal filtration is performed using 100 kDa filtration tubes; and C) the centrifugal filtration is repeated between about 5-10 times.

Provided herein are exemplary parameters (e.g., concentration, flow rate, temperature, filtration rate, etc.) for various techniques and processes utilized in one or more methods of liposome synthesis. As would be understood by one of ordinary skill in the art, any discrete parameter described herein is exemplary in nature, and does not limit the scope of the disclosure, as many discrete values of various parameters are contemplated by the present disclosure.

In various embodiments, the present disclosure provides a method for making active agent-encapsulating small unilamellar liposomes including: A) mixing an organic solvent and at least one lipid component to create a first solution; B) heating the first solution to a first temperature within a predetermined range of a transition temperature of the at least one lipid component; C) obtaining a second solution including: 1) an aqueous component; and 2) an active agent; D) heating the second solution to the first temperature; E) injecting the first solution into the second solution to form a mixture, wherein the at least one lipid component forms small unilamellar liposomes within the mixture; F) incubating the mixture at the first temperature for a predetermined period of time, wherein: 1) incubating the mixture causes encapsulation of the active agent by the small unilamellar liposomes; and 2) the incubated mixture includes active-agent encapsulating liposomes; and G) concentrating the mixture via centrifugal filtration, wherein concentrating the mixture removes a substantial portion of non-encapsulated active agent from the mixture.

According to various embodiments, the transition temperature may be about 55 degrees Celsius and the predetermined range is between about 0-10 degrees Celsius.

According to various embodiments, the first solution may include the organic solvent at a concentration between about 5-10%.

According to various embodiments, the organic solvent may be isopropyl alcohol.

According to various embodiments, injecting may occur at a rate of about 400 μL/min.

According to various embodiments, injecting may further include: A) vortexing, at a predetermined rate, the second solution immediately before and throughout injecting the first solution into the second solution; and B) vortexing, at the predetermined rate, the mixture that is formed after injecting the first solution.

According to various embodiments, the predetermined rate may be between about 400-800 revolutions per minute.

According to various embodiments, mixing the first solution may include vortexing the organic solvent at the predetermined rate immediately before, throughout and immediately after adding the at least one lipid component.

According to various embodiments, present methods may further include cooling the mixture to a second temperature that is below the transition temperature, wherein the cooled mixture includes active agent-encapsulating small unilamellar liposomes.

According to various embodiments, the second temperature may be about 0-5 degrees Celsius.

According to various embodiments, the predetermined period of time may be between about 1-2 hours.

According to various embodiments, present methods may further include concentrating the first solution via centrifugal filtration, wherein centrifugal filtration may be performed at a rate of about 6000 g and repeated between about 5-10 times.

According to various embodiments, centrifugal filtration may be further performed using 100 kDa filtration tubes.

According to various embodiments, the at least one lipid component may be DSPC.

According to various embodiments, the first solution may further include cholesterol and the cholesterol may also form the small unilamellar liposomes.

In various embodiments, the present disclosure provides a method for making active agent-encapsulating small unilamellar liposomes including: A) mixing an isopropyl alcohol at a concentration between 5-10%, at least one lipid component and cholesterol to create a first solution, wherein the at least one lipid component and the cholesterol form small unilamellar liposomes within the first solution, wherein mixing further includes: 1) vortexing the isopropyl alcohol, at a rate between about 400-800 revolutions per minute, immediately before and throughout adding the at least one lipid component and the cholesterol; and 2) vortexing the first solution at a rate between about 400-800 revolutions per minute; B) heating the first solution to a first temperature between about 45-65 degrees Celsius; C) concentrating the first solution by performing centrifugal filtration, wherein: 1) the centrifugal filtration is performed at a rate of about 6000 g; 2) the centrifugal filtration is performed using 100 kDa filtration tubes; and 3) the centrifugal filtration is repeated between about 5-10 times; D) obtaining a second solution including: 1) an aqueous component; and 2) an active agent; E) heating the second solution to the first temperature; F) injecting the first solution into the second solution at a constant rate of about 400 μL/min to form a mixture, wherein injecting further includes: 1) vortexing, at a predetermined rate between about 400-800 revolutions per minute, the second solution immediately before and throughout injecting the first solution into the second solution; and 2) vortexing, at the predetermined rate, the mixture that is formed after injecting the first solution; G) incubating the mixture at the first temperature for about 1-2 hours, wherein incubating the mixture causes encapsulation of the active agent by the small unilamellar liposomes; H) cooling the mixture to between about 0-5 degrees Celsius, wherein the cooled mixture includes active agent-encapsulating small unilamellar liposomes; and I) concentrating the mixture by performing centrifugal filtration, wherein concentrating the mixture removes a substantial portion of non-encapsulated active agent from the mixture, wherein: 1) the centrifugal filtration is performed at a rate of about 6000 g; 2) the centrifugal filtration is performed using 100 kDa filtration tubes; and 3) the centrifugal filtration is repeated between about 5-10 times.

In various embodiments, the present disclosure provides a liposomal solution including small unilamellar liposomes, wherein the small unilamellar liposomes may include: A) a polydispersity of less than about 0.40; B) a concentration, in the liposomal solution, between about 100 μM and 10 M; and C) a mean diameter less than about 140 nm.

According to various embodiments, the small unilamellar liposomes may further include an active agent that is encapsulated within the small unilamellar liposomes.

In various embodiments, the present disclosure provides a method for making liposomes with low polydispersities, with low diameters, and with tunable potencies that does not include secondary processing, including: A) mixing an alcohol solution of a charged lipid and an alcohol solution of cholesterol in a 2:1 molar ratio to form a mixed lipid solution including lipids, wherein the concentration of the lipids ranges between about 10M and about 100 microM; and B) injecting, at a constant rate, the mixed lipid solution into an aqueous solution, heated to a temperature within 0-10 degrees of the phase transition temperature of the charged lipid molecule, to form a liposome solution having an alcohol concentration of between 5% to 10%, wherein the liposome solution is shaken or vortexed during and after the time of injecting.

According to various embodiments, the alcohol of the alcohol solution may be isopropyl alcohol, the charged lipid may be DSPC, the transition temperature may be about 55° C., the constant rate may be 400 μL/min, the aqueous solution may include at least one active agent that is encapsulated by liposomes formed in the aqueous solution, and the liposomes may include a polydispersity of less than about 0.40 and a mean diameter less than about 140 nm.

### BRIEF DESCRIPTION OF THE FIGURES

FIG. 1 is a flowchart illustrating an exemplary process for synthesizing drug-encapsulating liposomes, according to one embodiment of the present disclosure.

FIG. 2 is a graph illustrating the effect of solvent on liposome formation, according to one embodiment of the present disclosure.

FIGS. 3A-B are graphs characterizing liposome synthesis, according to one embodiment of the present disclosure.

FIG. 4 A-B are graphs illustrating filtration effects and removal of non-encapsulated molecules, according to one embodiment of the present disclosure.

FIG. 5 is an exemplary image set of cells that were incubated with either filtered CMFDA-encapsulated liposomes or filtered CMFDA-doped liposomes, according to one embodiment of the present disclosure.

FIG. 6A-B are graphs illustrating evaluations of exemplary liposome filtration methods described herein, according to one embodiment of the present disclosure.

FIG. 7 is a graph illustrating evaluations of exemplary liposome synthesis solvents described herein, according to one embodiment of the present disclosure.

FIG. 8A-D are graphs illustrating evaluations of exemplary liposome encapsulation methods described herein, according to one embodiment of the present disclosure.

FIG. 9 is a graph illustrating exemplary liposome synthesis techniques as related to concentrations of a lipid component, according to one embodiment of the present disclosure.

FIG. 10 illustrates an exemplary liposome synthesis technique, according to one embodiment of the present disclosure.

FIG. 11 illustrates an exemplary technique for synthesizing targeted liposomes, according to one embodiment of the present disclosure.

FIG. 12 is a graph illustrating evaluations of exemplary liposome synthesis and encapsulation techniques, according to one embodiment of the present disclosure.

FIG. 13 is a graph illustrating evaluations of exemplary liposome synthesis and encapsulation techniques, according to one embodiment of the present disclosure.

FIG. 14 illustrates an exemplary technique for synthesizing active agent-encapsulating liposomes, according to one embodiment of the present disclosure.

FIG. 15 is an exemplary image set of liposomes synthesized by the present methods, according to one embodiment of the present disclosure.

FIG. 16 is an exemplary image illustrating effects of filtration techniques, according to one embodiment of the present disclosure.

FIG. 17 is a diagram of an exemplary liposome and liposome metrics, according to one embodiment of the present disclosure.

FIG. 18 is a graph illustrating effects of an exemplary filtration method on lipid concentration, according to one embodiment of the present disclosure.

FIG. 19 is a graph illustrating effects of an exemplary filtration method on lipid concentration, according to one embodiment of the present disclosure.

FIG. 20 is an exemplary image of small unilamellar liposomes synthesized using present methods, according to one embodiment of the present disclosure.

FIG. 21 is a graph illustrating effects of an exemplary liposome preparation method, according to one embodiment of the present disclosure.

FIG. 22 is a graph illustrating evaluations of exemplary liposome synthesis temperatures as related to liposome diameter, according to one embodiment of the present disclosure.

FIGS. 23A-B are graphs illustrating evaluations of membrane dynamics in response to thermal equilibration, according to one embodiment of the present disclosure.

FIG. 24 is an exemplary image of a membrane dynamic simulation and depicts adsorption of DXR to a lipid membrane, according to one embodiment of the present disclosure.

FIG. 25 is a graph illustrating evaluations of exemplary interactions between DSPC and DXR, according to one embodiment of the present disclosure.

FIG. 26 is an exemplary diagram of a DXR-lipid interaction, according to one embodiment of the present disclosure.

FIGS. 27A-C are graphs illustrating evaluations of exemplary lipids, DXR and DXR-lipid interactions, according to one embodiment of the present disclosure.

FIG. 28 is an exemplary diagram set illustrating structural features of a liposome, according to one embodiment, according to one embodiment of the present disclosure.

FIGS. 29 A-B are graphs illustrating evaluations of exemplary DXR-lipid electrostatic energy and interaction energy at various temperatures, according to one embodiment of the present disclosure.

FIGS. 30 A-C are graphs and an exemplary image set illustrating evaluations of thermal equilibration as a universal encapsulation method, according to one embodiment of the present disclosure.

FIG. 31 is a graph illustrating an evaluation of hydrogen bonds between DSPC and DOX, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

Disclosed herein are methods for synthesis and purification of injectable nanocarriers (SPIN) for rapid and efficient production of small drug-loaded liposomes, which may be accomplished using common benchtop equipment. In various embodiments, the present methods may provide for solutions including unilamellar liposomes, with mean a diameter of 80 nm and a polydispersity of 0.13. In at least one embodiment, the present methods provide techniques for liposome synthesis that do not require any secondary post-processing techniques. In various embodiments, the presents methods may provide liposomes that encapsulate active agents (for example, dextrans [300-20,000 Da]), representing small and large molecular drug formulations, without significantly affecting one or more characteristics of the liposomes. In one or more embodiments, the present methods provide techniques for removing non-encapsulated molecules from liposomal solutions by using a filter centrifugation step. In at least one embodiment, filter centrifugation may largely eliminate necessities for tedious ultracentrifugation protocols (e.g., to concentrate and purify liposomal solutions). In one or more embodiments, functional efficacy of loaded liposomes as drug delivery vehicles may be validated by encapsulating a fluorescent cell tracker (CMFDA) and observing the liposomal release and subsequent uptake of dye by metastatic breast cancer cells (MDA-MB-231) in vitro. Thus, the disclosed systems, methods and compositions address existing challenges associated with liposome preparation in resource-limited settings and offer significant potential for advances in translational pharmaceutical development.

Several factors including the concentration and choice of reagents (lipids, cholesterol, and alcohols), synthesis temperature, injection method, and the choice of encapsulated drug can influence the characteristics of liposomes. Addressing these limitations, disclosed herein is a method for rapidly synthesizing, concentrating, and purifying drug encapsulated SUL's using common laboratory equipment. In various embodiments, liposomes containing fluorescent molecules of known molecular weights were produced using an injection method. In at least one embodiment, non-encapsulated fluorophores were then removed via benchtop filter centrifugation, using a molecular weight cut-off filter that prevents loss of liposomes while ensuring the removal of free small molecules. Also disclosed herein are methods for drug delivery as shown, in various embodiments, by exposing cells to liposomes containing 5-chloromethylfluorescein diacetate (CMFDA), a marker that becomes fluorescent upon being hydrolyzed by esterases in the cytoplasm of a cell. Methods and compositions disclosed herein offer the potential to facilitate the discovery of novel liposomal formulations for a wider implementation in point-of-care clinical settings.

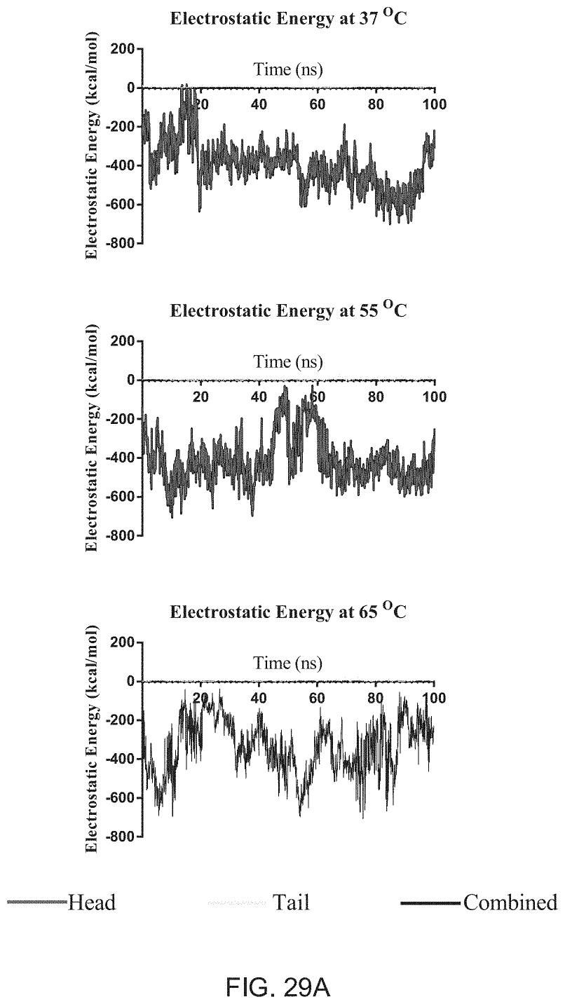

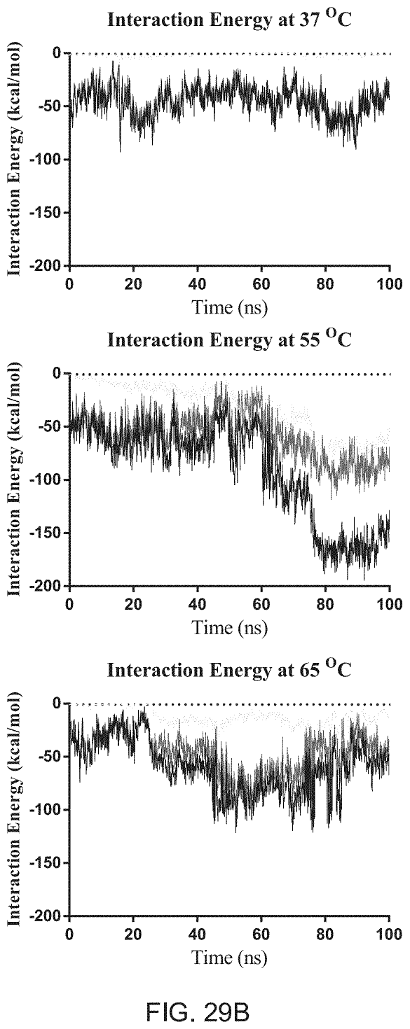

Disclosed herein are methods for making liposomes that have low polydispersity and high carrier capacity. Liposomes are generally made from charged lipids and other lipids. For example, charged lipids such as phospholipids are the usually the main component of the liposome's membrane. The phospholipids used in liposomes are further categorized into natural and synthetic phospholipids. An exemplary phospholipid used is known as lecithin (also known as phospatidylocholine) and is amphipathic. Other lipids used to make liposomes include cholesterol and 1, 2-distearoyl-sn-glycero-3-phosphocholine (“DSPC”). In at least one embodiment, cholesterol molecules in a lipid membrane may increase separation between charged, e.g. choline, head groups of phospholipids (e.g., that form the membrane), which reduce the normal hydrogen bonding and electrostatic interaction (as is illustrated in FIGS. 29A-B). In various embodiments, liposomes comprising cholesterol and DSPC are disclosed herein, but the disclosure is not to be limited to liposomes comprising only these lipids, liposomes formed from these and other charged and neutral lipids are contemplated. In at least one embodiment, a method for synthesizing liposomes including DSPC and cholesterol may include mixing in a 2:1 molar ratio a lipid solution of DSPC and a lipid solution of cholesterol, each dissolved in alcohol. In one or more embodiments, mixing may be accompanied by heating and shaking to ensure complete mixture of the two solutions to form an alcoholic mixed lipid solution. In at least one embodiment, the mixed lipid solution may be injected into an aqueous solution at a constant rate while the aqueous solution is moved, for example, is vortexed or shaken. For example, the mixed lipid solution may be injected at a rate of 50 μL/minute, and an aqueous solution may be moved, e.g., vortexed at speeds of from 400 rpm to 800 rpm, and speeds thereinbetween. For example, the aqueous solution may be vortexed at 600 rpm. In one or more embodiments, after injection of the mixed lipid solution, vortexing may continue for a time, at the same or a speed different from that of the speed used during injection.

In various embodiments, type of alcohol (or other solvent) used in forming the lipid solution and the lipid concentration may affect the size of the liposomes formed and the polydispersity of the liposomes formed. For example, isopropyl alcohol in the lipid solution may be more effective than ethanol. In at least one embodiment, final alcohol concentration may influence size and polydispersity of synthesized liposomes. For example, in adding the mixed lipid solution into the aqueous solution, a final alcohol concentration of 10% may yield smaller (lower diameter), less disperse liposome particles than lower alcohol concentrations. In various embodiments, while maintaining a DSPC:cholesterol ratio of 2:1, in a 10% final isopropyl alcohol concentration, lipid concentration may be provided in a range from 10 M to 100 microM. In one embodiment, after liposomes are formed in a final mixture of aqueous solution and mixed lipid solution, the liposomes may be rinsed and/or filtered to remove unreacted lipids, alcohol, the aqueous solution or unencapsulated active agents. In one or more embodiments, a filtration-type centrifugation step may be used for this rinsing step, but the methods disclosed herein do not comprise an exclusion centrifugation step that separates the formed liposomes into particularly sized compositions. Instead, the liposomes that are formed may be separated from other components used in the methods disclosed (for example, non-encapsulated agent).

In at least one embodiment, increasing a temperature of the aqueous solution above room temperature or a lipid transition temperature may aid in decreasing the size and to lower the polydispersity of the liposomes formed in the above method. In some embodiments, below a transition temperature of 37° C., liposomes may be highly polydisperse with diameter sizes of approximately 200 nm. In some embodiments, at temperatures near the phase transition temperature of the charged lipid molecule, lower diameter size and lower polydispersity may be found. In various embodiments, the temperatures near the phase transition temperature of a charged lipid molecule may refer to a temperature that is between 0-20 degrees above or below the phase transition temperature of the charged lipid molecule.

In one or more embodiments, injection flow rate of the mixed lipid solution into the aqueous solution may also affect liposome size and polydispersity. For example, flow rates of 25 microL/min. to 400 microL/min may result in liposomes with small diameters and low polydispersity. In some embodiments, at higher flow rates, liposome size and polydispersity may increase.

In various embodiments, active agents may be added to the aqueous solution to form liposomes that encapsulate active agents and, thus, that are effective in drug delivery. In at least one embodiment, one or more active agents may be added to an aqueous solution to result in active agent-encapsulating liposomes. As used herein, an “active agent” may be any compound, molecule or biological molecule (e.g., a protein that is capable of activity in a biological or chemical environment). Active agents may include, but are not limited to, drugs, chemical compounds, biologics, chemical elements, vitamins, antibiotics, chemotherapeutic agents, herbicides, pesticides, growth enhancers, growth retardants, and biosimilar molecules.

Disclosed herein are methods of using liposomes comprising at least one active agent for delivery of at least one active agent to cells, comprising contacting at least one cell to an active-agent comprising liposome made by methods disclosed herein. In one embodiment, a method of drug delivery may include exposing at least one cell to an active-agent comprising liposome made by methods disclosed herein. In one or more embodiments, a method of treatment of a physiological condition in a subject may include contacting at least one cell of the subject to an active agent-encapsulating liposome made by methods disclosed herein. As used herein, a physiological condition may refer to a physiological state of a living organism, and the condition may be a normal, abnormal, pathological, deleterious, chronic or acute condition of at least one cell of a subject. A subject comprises any living or dying organism found on Earth.

### DETAILED DESCRIPTION OF VARIOUS FIGURES

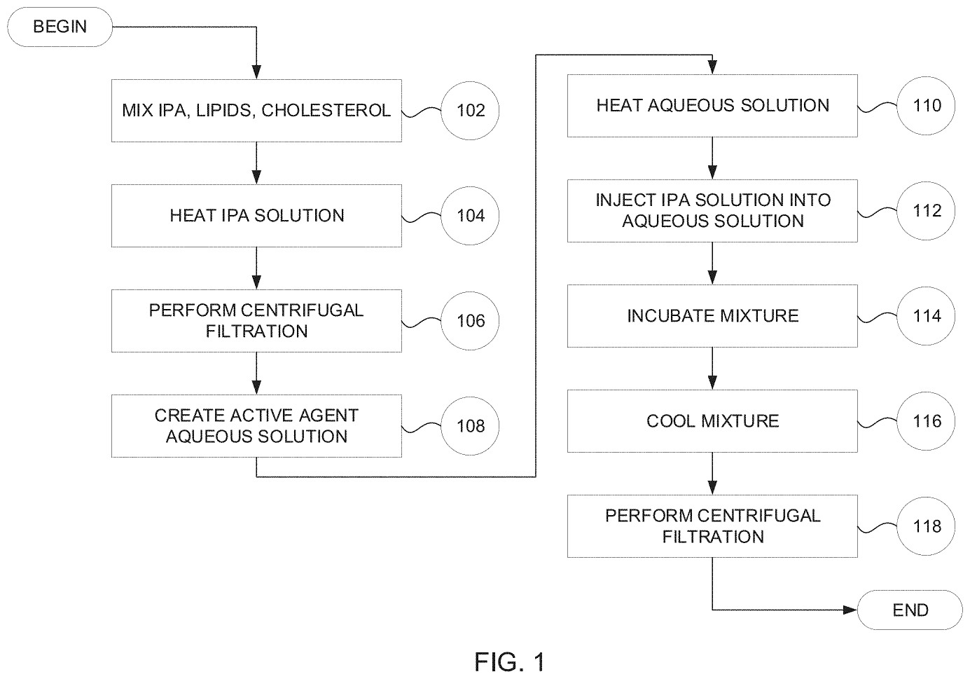

FIG. 1 is a flowchart illustrating an exemplary method for synthesizing active-agent encapsulating, small unilamellar liposomes. As will be understood by a person having ordinary skill in the art, the steps and process show in FIG. 1 (and those of all other flowcharts and sequence diagrams shown and described herein) may operate concurrently and continuously, are generally asynchronous and independent, and are not necessarily performed in the order shown.

At step 102, a first liposomal is formed by mixing isopropyl alcohol (“IPA”), cholesterol, a lipid component (for example, DSPC) and a sufficient aqueous portion. In various embodiments, the cholesterol and the lipid component may form liposomes upon being deposited into the first solution. In one or more embodiments, the IPA may increase the permeability of the liposomes. In at least one embodiment, the cholesterol and the liquid component may have each been stored in alcohol solutions prior to being deposited into the IPA. In one or more embodiments, the ratio of the lipid component and the cholesterol (in the first liposomal solution) may be between about 1:1 and about 10:1. In some embodiments, the ratio of the lipid component and the cholesterol may be about 2:1. In various embodiments, a concentration of the lipid component may be between about 100 μM to 10 M. In one or more embodiments, the IPA may be at a concentration (in the first liposomal solution) between about 5-20% (v/v). In various embodiments, the IPA may be at a concentration of about 10% (v/v).

In at least one embodiment, the IPA (and, later, the first liposomal solution) may be agitated and/or vortexed throughout formation of the first liposomal solution. In some embodiments, vortexing may be performed at a rate between about 100-1000 revolutions per minute (RPM). In one embodiment, vortexing may be performed at a rate between about 400-800 revolutions per minute.

At step 104, the first liposomal solution is heated to a particular temperature within a predetermined range of a transition temperature of the lipid component. In at least one embodiment, the transition temperature may be about 37° C. and the predetermined range may be about 0-20 degrees. In at least one embodiment, the particular temperature may be about 55° C. In various embodiments, heating the first liposomal solution may further increase permeability of the liposomes therein (e.g., to further facilitate active agent encapsulation). In one or more embodiments, the first solution may be maintained at the particular temperature upon formation (of the first solution) and prior to being injected into an active agent solution (e.g., as described herein). In at least one embodiment, the particular temperature may be a predetermined temperature that is above room temperature.

At step 106, the first liposomal solution is concentrated via centrifugal filtration. In one or more embodiments, centrifugal filtration may remove cholesterol and the lipid component that were not incorporated into liposomes. In at least one embodiment, the centrifugal filtration may be performed at a rate between about 1,000-10,000 g. In one embodiment, the centrifugal filtration may be performed at a rate of about 6,000 g. In various embodiments, the centrifugal filtration may be performed using filter tubes including a filtration metric between about 10-200 kDa. In one embodiment, the centrifugal filtration may be performed using 100 kDa filter tubes. In some embodiments, the centrifugal filtration may be performed for a predetermined time period between about 5-60 minutes. In at least one embodiment, the predetermined time period may be about 10 minutes. In one or more embodiments, the centrifugal filtration may be performed in 2-10 repetitions. In at least one embodiment, each of the centrifugal filtration repetitions may be performed for about 10-15 minutes.

At step 108, an aqueous active agent solution may be created (or, in some embodiments, obtained). In at least one embodiment, the active agent solution may include an active agent that includes, but is not limited to: 1) drugs; 2) chemical compounds; 3) biologics; 4) chemical elements; 5) vitamins; 6) antibiotics; 7) chemotherapeutic agents; 8) herbicides; 9) pesticides; 10) growth enhancers; 11) growth retardants; and 12) biosimilar molecules. In various embodiments, the active agent may be lipophilic.

At step 110, the active agent solution may be heated to a particular temperature within a predetermined range of the transition temperature of the lipid component. In at least one embodiment, the transition temperature may be about 37° C. and the predetermined range may be about 0-20 degrees. In at least one embodiment, the particular temperature may be about 55° C. In one or more embodiments, the active agent solution may be maintained at the particular temperature upon formation and prior to being injected with the first liposomal solution (e.g., as described herein).

At step 112, the heated first liposomal solution is injected into the heated aqueous solution to form a second liposomal solution. In one or more embodiments, injection may be performed at a constant rate. In various embodiments, the constant rate may be between about 10-1000 μL/min. In at least one embodiment, the constant rate may be about 400 μL/min. In at least one embodiment, the active agent solution (and, upon formation, the second liposomal solution) may be agitated and/or vortexed throughout formation of the second liposomal solution. In some embodiments, vortexing may be performed at a rate between about 100-1000 revolutions per minute (RPM). In one embodiment, vortexing may be performed at a rate between about 400-800 revolutions per minute.

In one or more embodiments, upon injection into the active agent solution, the liposomes (of the first liposomal solution) may encapsulate particles of the active agent included therein. Thus, in at least one embodiment, the second liposomal solution includes active agent-encapsulating liposomes.

At step 114, the second liposomal solution is incubated at a specific temperature and for a specific time interval. In various embodiments, incubation of the second liposomal solution may increase encapsulation of the active agent by the liposomes (e.g., present in the second liposomal solution). In at least one embodiment, incubation may be referred to as “thermal equilibration”, wherein the second liposomal solution may be incubated until the liposomes therein have reached a maximum encapsulation efficiency with regard to the active agent (also present therein).

In one or more embodiments, the specific temperature may be within a predetermined range (e.g., 0-20 degrees) of the transition temperature of the lipid component. In various embodiments, the specific temperature may be about 30-60° C. In one embodiment, the specific temperature may be about 37° C. In some embodiments, the specific temperature may be about 55° C. In some embodiments, the specific temperature may be a predetermined temperature that maximizes encapsulation activity (by the liposomes). In various embodiments, the specific time interval may be a predetermined time interval that allows thermal equilibration to proceed to completion. In at least one embodiment, the specific time interval may between about 1-5 hours. In one embodiment, the specific time interval may be about 2 hours.

At step 116, the second liposomal solution may be cooled. In at least one embodiment, the second liposomal solution may be cooled to a temperature that is below the transition temperature of the lipid component. In one or more embodiments, the second liposomal solution may be cooled to a temperature between about 0-5° C. In on embodiment, the second liposomal solution may be cooled to about 4° C. In at least one embodiment, cooling may be performed to reduce permeability of the active agent-encapsulating liposomes (present within the second liposomal solution).

At step 118, the second liposomal solution may be concentrated via centrifugal filtration. In one or more embodiments, the centrifugal filtration may remove one or more non-encapsulated active agent components (from the second liposomal solution), thereby increasing concentration of the active agent-encapsulating liposomes in the second liposomal solution. In at least one embodiment, the centrifugal filtration may be performed at a rate between about 1,000-10,000 g. In one embodiment, the centrifugal filtration may be performed at a rate of about 6,000 g. In various embodiments, the centrifugal filtration may be performed using filter tubes including a filtration metric between about 10-200 kDa. In one embodiment, the centrifugal filtration may be performed using 100 kDa filter tubes. In some embodiments, the centrifugal filtration may be performed for a predetermined time period between about 5-60 minutes. In at least one embodiment, the predetermined time period may be about 10 minutes. In one or more embodiments, the centrifugal filtration may be performed in 2-10 repetitions. In at least one embodiment, each of the centrifugal filtration repetitions may be performed for about 10-15 minutes. In various embodiments, the filtered second liposomal solution may include active agent-encapsulating, small unilamellar liposomes with an average diameter between about 70-150 nm and a polydispersity below 0.4. In at least one embodiment, average diameter may be about 80-120 nm and the polydispersity may be about 0.13.

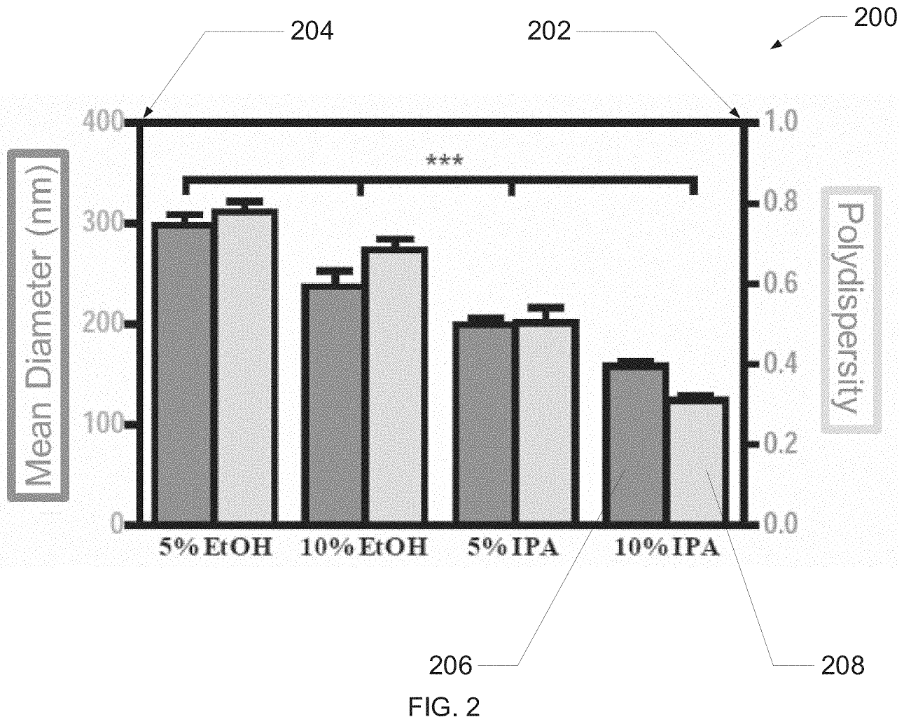

FIG. 2 illustrates a bar graph 200 that relates alcohol concentration and alcohol type in a first liposomal solution (as described herein) to polydispersity 202 and mean diameter 204. Thus, the graph 200 demonstrates relationships between the type and concentration of alcohol (in a first liposomal solution) and the mean diameter and polydispersity of liposomes formed therein. The graph 200 provides a 10% IPA diameter bar 206 and a 10% IPA polydispersity bar 208. As can be observed in the graph 200, the 10% IPA diameter 206 and the 10% IPA polydispersity 208 minimize the values of mean diameter 204 and polydispersity 202 (in comparison to other data therein). Thus, in one or more embodiments, the graph 200 indicates that a first liposomal solution including 10% IPA may present liposomes having a low polydispersity and minimized average diameter (in comparison to liposomes produced in solutions of alternative alcohol content).

Thus, in at least one embodiment, the choice of alcohol solvent can affect vesiculation of synthesized liposomes. For example, liposomes were synthesized using either EtOH or IPA with final concentrations of 5% or 10%. In the same example, resulting solutions were analyzed using DLS (e.g., wherein all DLS data was obtained by triplicate measurements of at least 3 independently synthesized suspensions (n≥3) and ANOVA with a Tukey post-test was used to determine the statistical significance between samples (***=p<0.0001). Continuing with the same example, liposomes synthesized under 10% IPA may demonstrate both lower diameters and polydispersities.

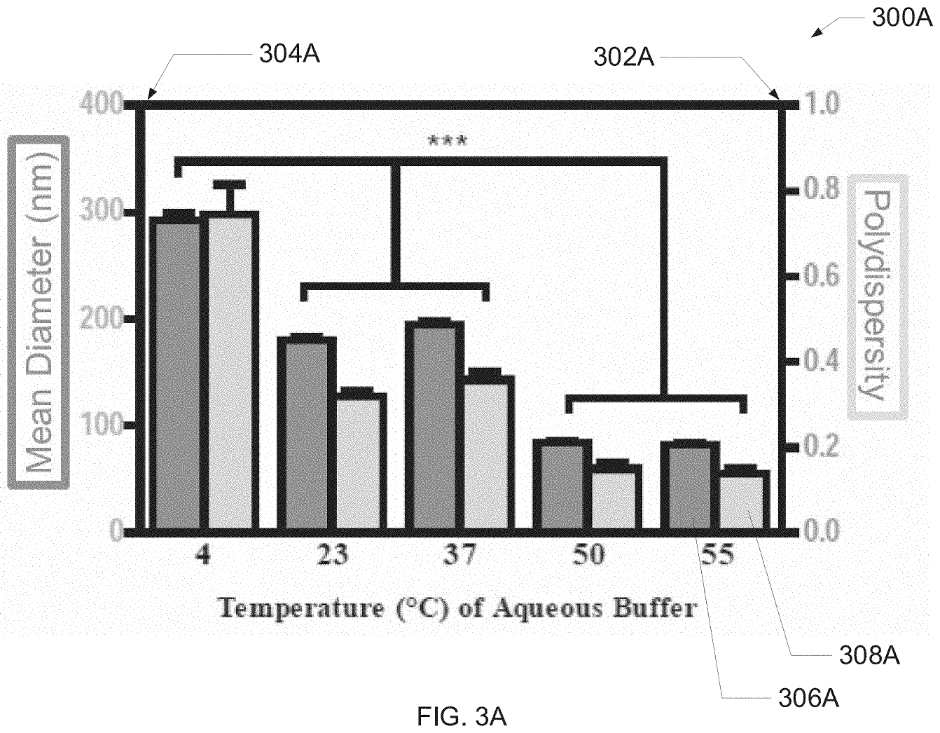

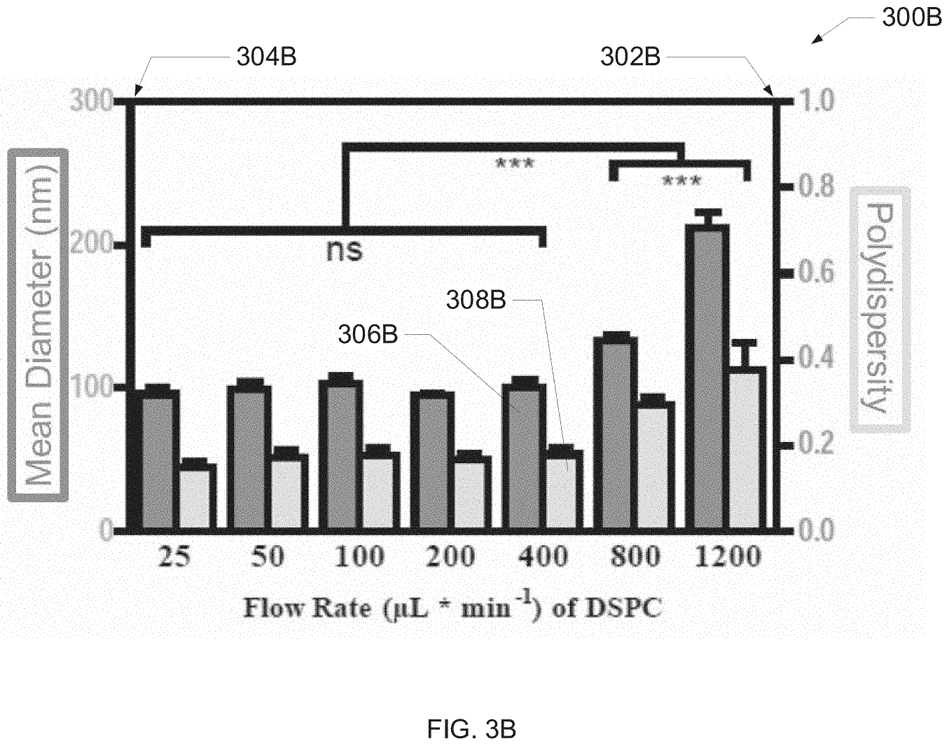

FIG. 3A illustrates a bar graph 300A that relates temperature of an aqueous liposome solution to polydispersity 302A and mean diameter 304A of liposomes present therein. Thus, the graph 300A demonstrates relationships between temperature (in an aqueous solution) and the mean diameter and polydispersity of liposomes formed therein. The graph 300A provides a 55° C. diameter bar 306A and a 55° C. polydispersity bar 308A. As can be observed in the graph 300A, the diameter bar 306A and the polydispersity bar 308A minimize the values of mean diameter 304A and polydispersity 302A (in comparison to other data therein). Thus, in one or more embodiments, the graph 300A indicates that a liposomal solution heated to 55° C. may present liposomes having a low polydispersity and minimized average diameter (in comparison to liposomes produced in solutions at lower temperatures).

FIG. 3B illustrates a bar graph 300B that relates injection flow rate (e.g., of an alcoholic liposomal solution into an aqueous solution) to polydispersity 302A and mean diameter 304B of liposomes formed in the aqueous solution. Thus, the graph 300B demonstrates relationships between injection flow rate (of a liposomal solution into an aqueous solution) and the mean diameter and polydispersity of active agent-encapsulating liposomes formed therein. The graph 300B provides a 400 μL/min diameter bar 306B and a 400 μL/min polydispersity bar 308B. As can be observed in the graph 300B, the diameter bar 306B and the polydispersity bar 308B minimize the values of mean diameter 304B and polydispersity 302B (in comparison to data of higher flow rates provided therein). Thus, in one or more embodiments, the graph 300B indicates that a liposomal solution injected into an aqueous solution of 400 μL/min may present liposomes having a low polydispersity and minimized average diameter (in comparison to liposomes produced in solutions, wherein injection occurred at a higher rate).

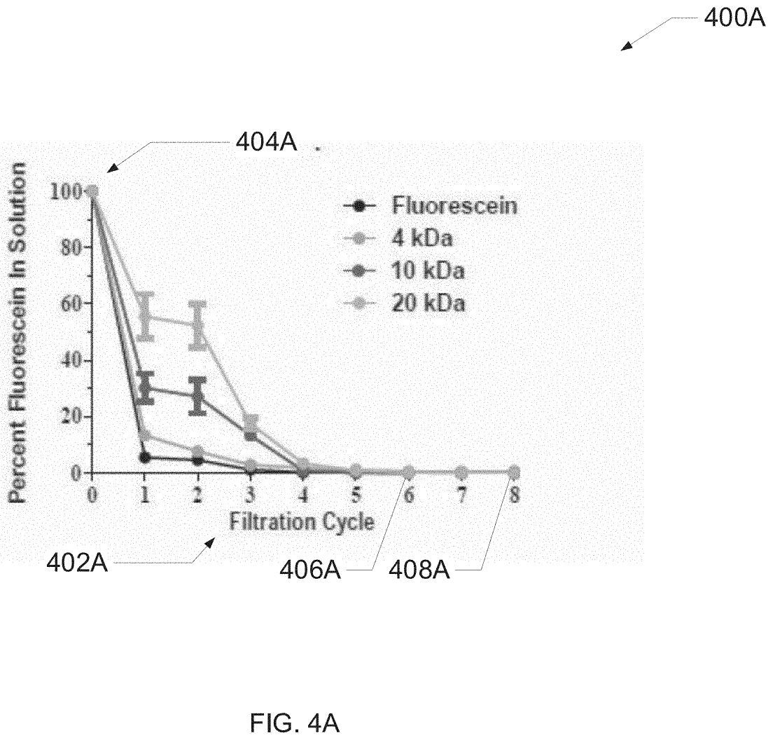

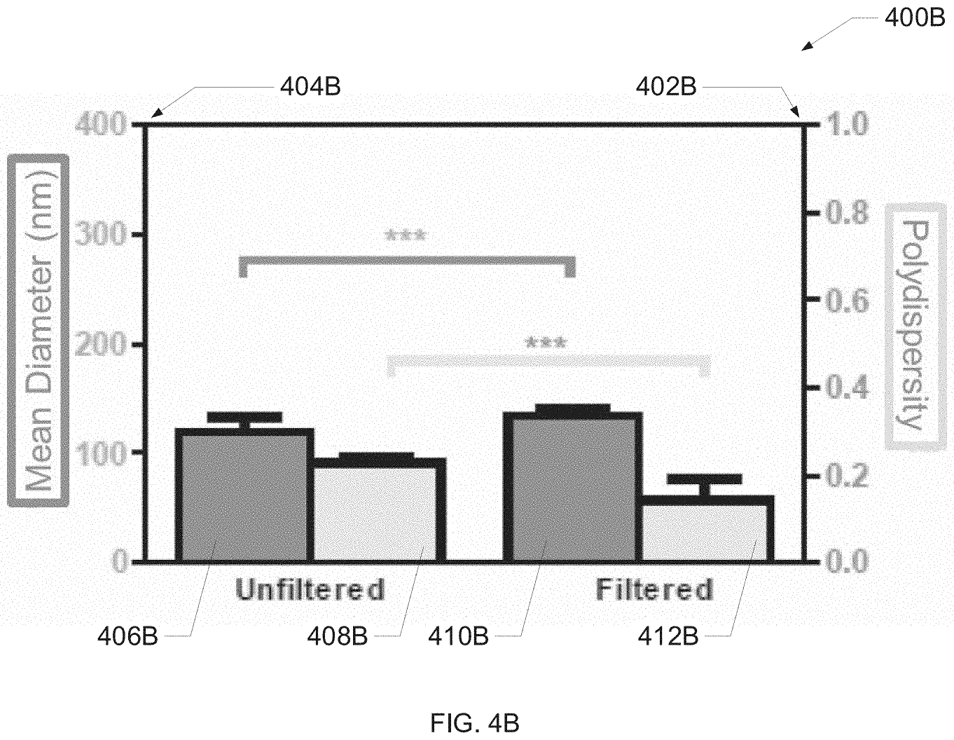

FIG. 4A illustrates a scatter plot 400A that relates centrifugal filtration repetition 402A to percent fluorescence 404A. In various embodiments, solutions containing liposomes and, initially, non-encapsulated fluorescein were filtered via centrifugal filtration to identify a number of filtration repetitions required to remove 99% and 99.9% of fluorescein from the solutions. Thus, the plot 400A demonstrates relationships between centrifugal filtration (of a liposomal solution containing non-encapsulated fluorescein) and percentage of fluorescein in the solution following each repetition (e.g., as measured via fluorescence intensity of each solution). As can be observed in the plot 400A at point 406A, 99% of fluorescein may be removed from a liposomal solution following 6 repetitions of centrifugal filtration. As can be further observed in the plot 400 at 408A, 99.9% of fluorescein may be removed from a liposomal solution following 8 repetitions of centrifugal filtration. Thus, in one or more embodiments, the plot 400A indicates that a liposomal solution may be filtered (via centrifugal filtration) in at least 6 repetitions to remove at least 99% of non-encapsulated fluorescein therein.

Thus, in various embodiments, a concentration of the non-encapsulated molecules may be reduced by 99% through 6 filtration cycles, and 99.9% through 8 filtration cycles, irrespective of the molecular weight. An exemplary protocol for evaluating concentration reduction may include performing all measurements in triplicates of at least six replicates (n≥6) and performing DLS measurements before and after filtration to ensure filtration has no effect on liposome characteristics and does not lead to aggregation of liposomes. An exemplary protocol may further include, but is not limited to, conducing triplicate measurements on 12 samples (n=12) and performing a t-test to determine statistical significance of differences between samples.

FIG. 4B illustrates a bar graph 400B that relates filtering of a liposomal solution to polydispersity 302A and mean diameter 304B of liposomes therein. Thus, the graph 400B demonstrates relationships between filtering (via centrifugal filtration) and the mean diameter and polydispersity of liposomes therein. The graph 300B provides an unfiltered diameter bar 406B and an unfiltered polydispersity bar 408B (e.g., associated with a solution prior to filtration), as well as a filtered diameter bar 410B and a filtered polydispersity bar 412B (e.g., associated with the solution following filtration). As can be observed in the graph 400B, the unfiltered diameter bar 406B and filtered diameter bar 410B are not significantly dissimilar. As can be further observed in the graph 400B, the unfiltered polydispersity bar 408B and the filtered polydispersity bar 412B are not significantly dissimilar. Thus, in one or more embodiments, the graph 400B indicates that centrifugal filtration may not significantly alter mean diameter and polydispersity of liposomes in a solution.

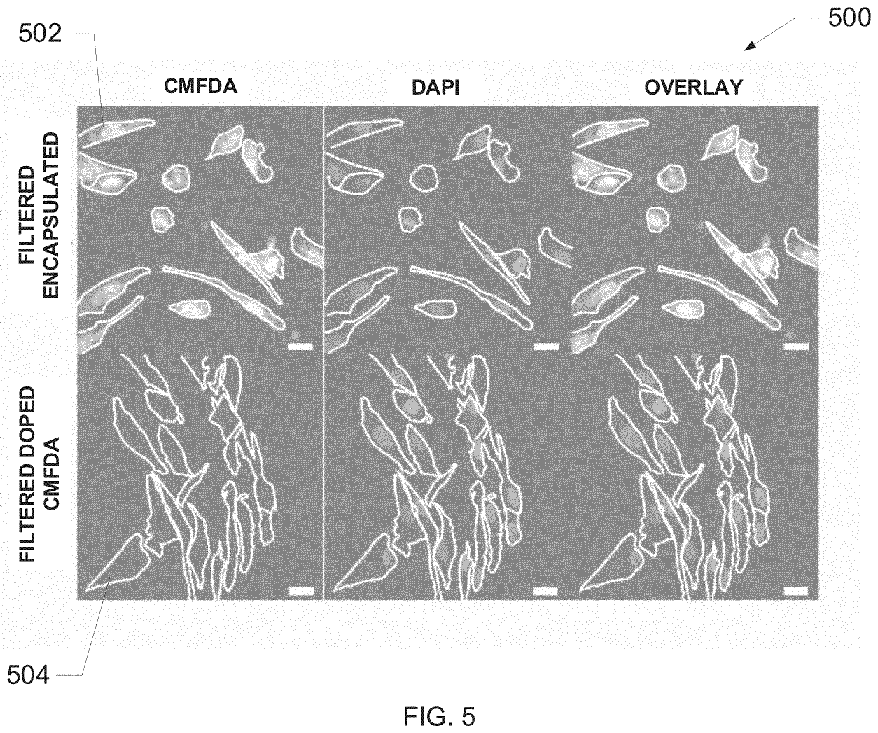

FIG. 5 illustrates an exemplary image set 500 of cells that were incubated with filtered CMFDA-encapsulated liposomes (e.g., liposomes produced via the present methods) and cells that were incubated with liposomes doped with CMFDA and subsequently filtered. The image set 500 includes filtered-encapsulated cell 502 and filtered-doped cell 504. In cell 502 and cell 504, presence of CMFDA within the cell may indicated by fluorescence (e.g., bright spots) in the stained image. The image set 500 indicates that cell 502 contained CMFDA, but cell 504 did not contain significant amounts of CMFDA. Accordingly, the image set 500 demonstrates that liposomes loaded with an active agent via encapsulation may be more effective in transporting an active agent (CMFDA in this case) into a cell than liposomes loaded with an active agent via doping.

In one or more embodiments, an exemplary protocol for obtaining the image set 500 may include taking confocal images of cells following 48 hours of incubation with filtered suspensions containing CMFDA (green) loaded liposomes or empty liposomes with CMFDA doped into the suspension. In at least one embodiment, the protocol may further include, but is not limited to, staining the cells (e.g., MDA-MB-231 cells) with DAPI (blue), tracing outlines of cells and providing (in images) a scale bars representing 20 μm.

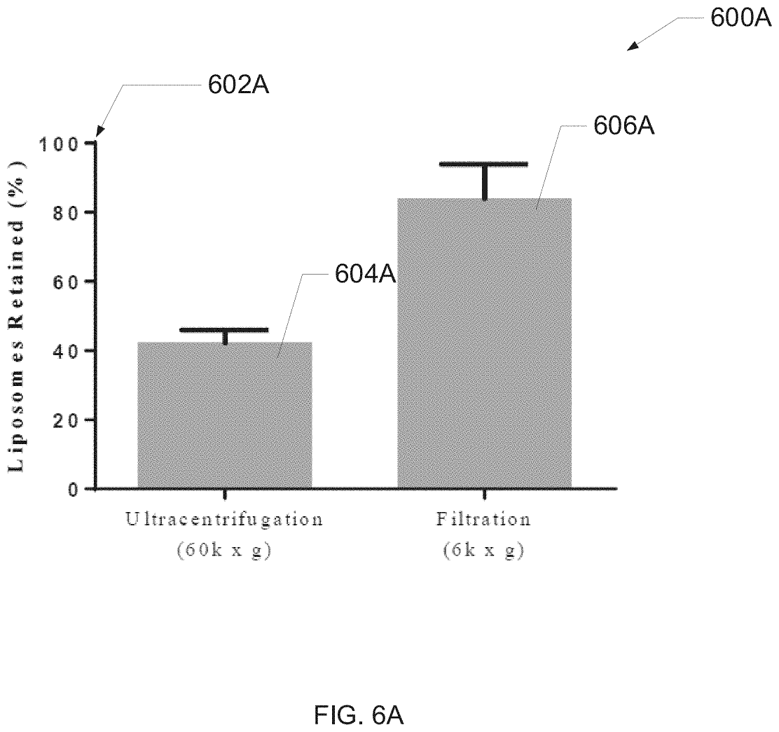

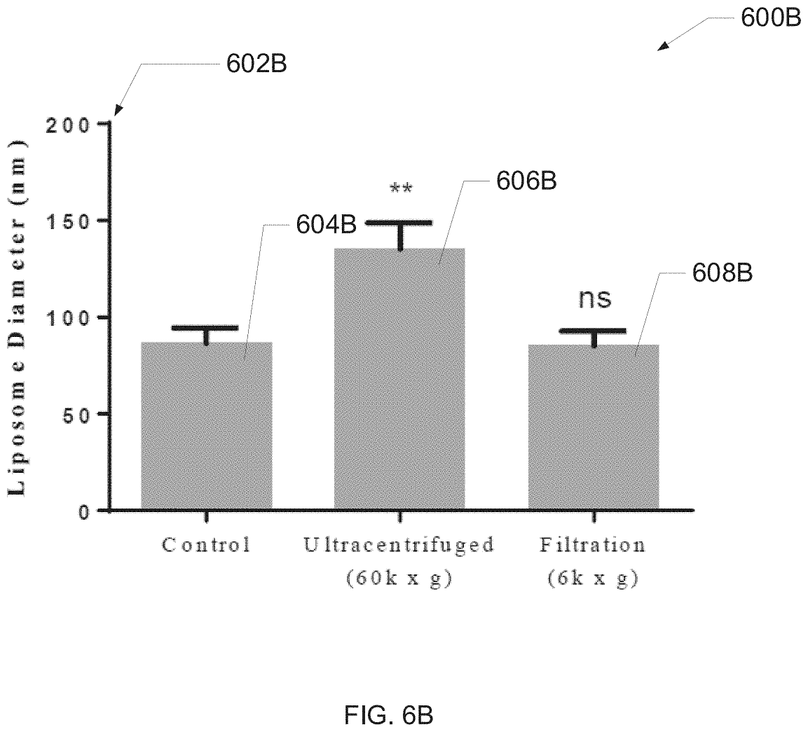

FIG. 6A illustrates a bar graph 600A that provides percentages of liposomes retained 602A in solutions that were filtered via either ultracentrifugation or centrifugal filtration. The graph 600A includes an ultracentrifugation bar 604A and a centrifugal filtration bar 606A. As can be observed in the graph 600A, the centrifugal filtration bar 606A demonstrates that centrifugal filtration methods yield solutions that retain a greater percentage of liposomes than solutions treated via ultracentrifugation methods. Thus, centrifugal filtration methods may be more efficient for preparing filtered and/or concentrated liposome solutions. In addition, ultracentrifugation methods typically require equipment that is more expensive than that required for centrifugal filtration. Accordingly, centrifugal filtration may also be comparatively advantageous with respect to both liposome conservation (in filtering) and equipment expenses.

FIG. 6B illustrates a bar graph 600B that provides average liposome diameters 602B of liposomes that were subjected to no treatment, filtering via ultracentrifugation or filtering via centrifugal filtration. The graph 600B includes a control bar 604B (e.g., wherein no filtration was performed on solution(s) represented therein), an ultracentrifugation bar 606B and a centrifugal filtration bar 608B. As can be observed in the graph 600A, the centrifugal filtration bar 608B is substantially similar in magnitude to the control bar 604B, while the ultracentrifugation bar 606B is substantially dissimilar from the other bars. Thus, the graph 600B indicates that: 1) ultracentrifugation may (undesirably) yield filtered solutions with elevated liposome diameters; and 2) centrifugal filtration my yield filtered solutions without significantly increasing diameters of liposomes therein.

Thus, in various embodiments, the present filtration process retains a population of liposomes efficiently (e.g., as seen in lipid size distribution and lipid concentration). In one or more embodiments, a resulting liposome population increases in diameter from 86.6 nm to 135.4 nm when ultracentrifuged (e.g., as a result of small liposomes remaining in the supernatant). In at least one embodiment, the change in diameter when filtered may not be statistically significant, insinuating the relative population was retained. In one or more embodiments, analysis of lipid concentration may demonstrate nearly 60% of the liposomes are lost following ultracentrifugation, whereas filtration may retain 84% of the total liposomes in solution.

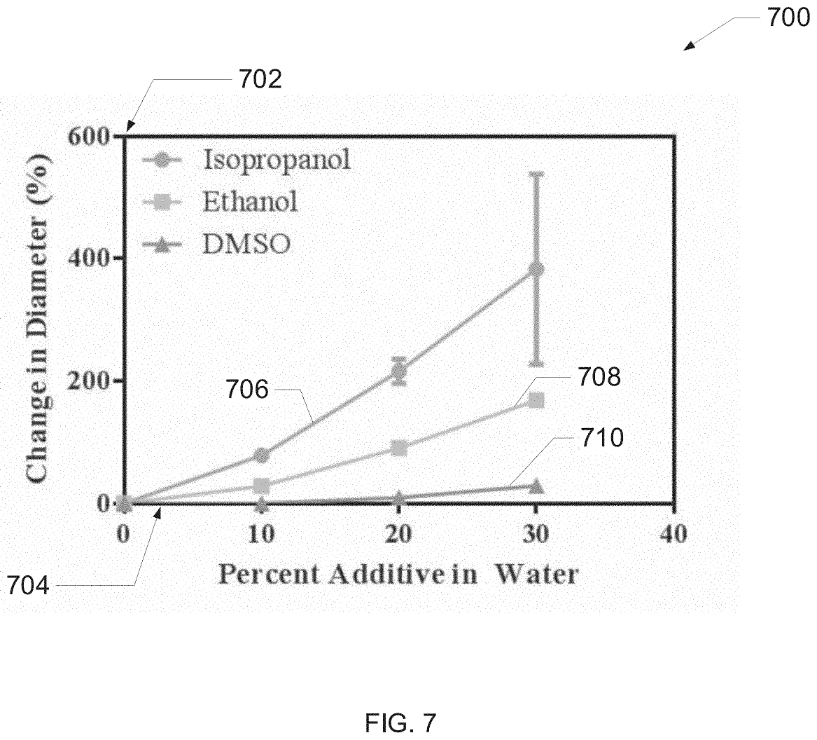

FIG. 7 illustrates a scatter plot 700 that relates percentage changes in liposome diameter 702 to percentages of various solvents 704 added to solutions containing liposomes. Thus, the plot 700 demonstrates relationships between diameter transformation and solvent type, and solvent percentage. The plot 700 includes an isopropanol trend line 706, an ethanol trend line 708 and a dimethyl sulfoxide (“DMSO”) trend line 710. As can be observed in the plot 700, the DMSO trend line 710 demonstrated (for any percentage 704 between 0-30%) the lowest percentage change to liposome diameter 702, and the isopropanol trend line 706 presented the greatest percentage change. Accordingly, the plot 700 indicates that DMSO may be a suitable alternative to both ethanol and isopropanol for use in liposomal formulations. In various embodiments, the present methods may substitute organic solvents (such as isopropanol) with DMSO (in suitable proportions described herein). In particular, the present methods may substitute an organic solvent for DMSO when producing liposomal solutions that include active agents whose activity may be compromised by the organic solvent.

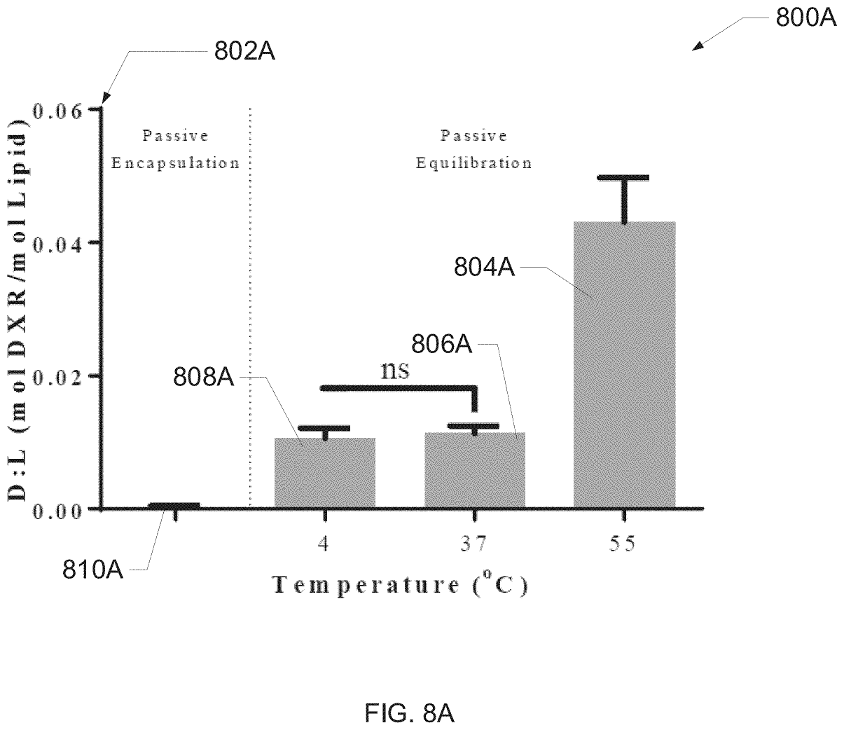

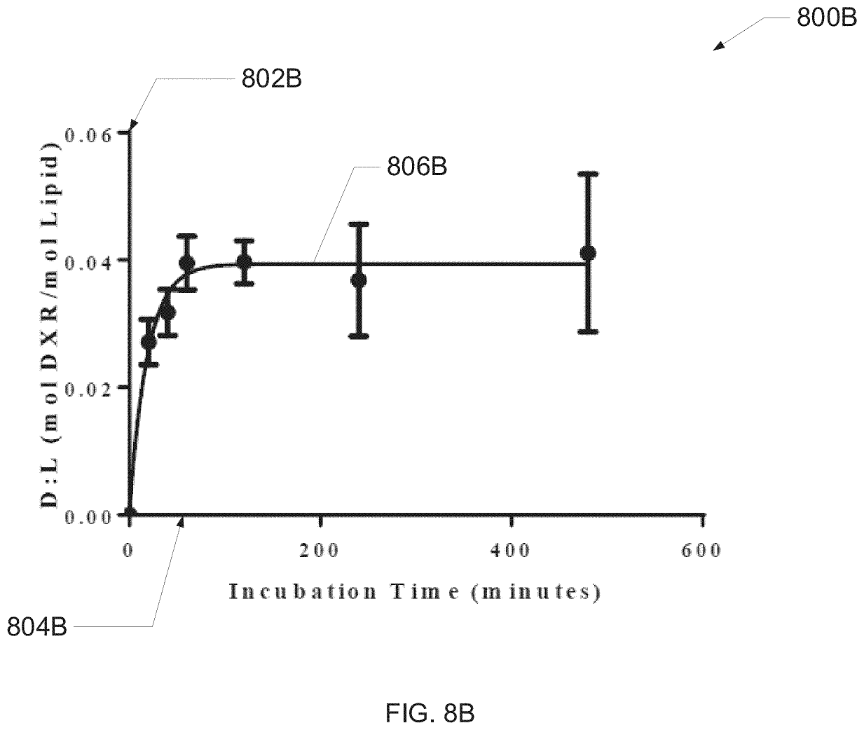

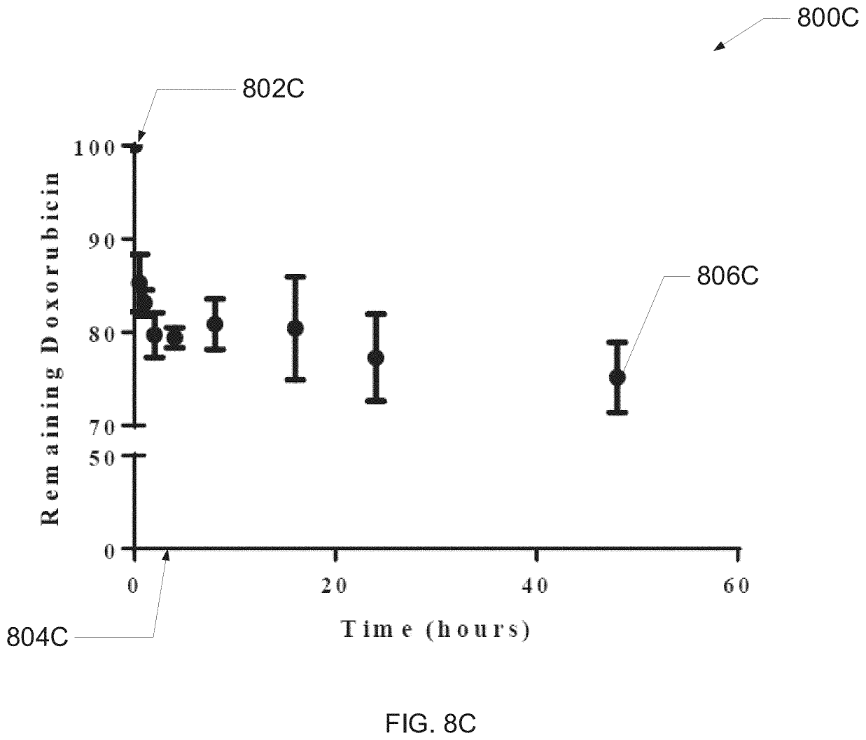

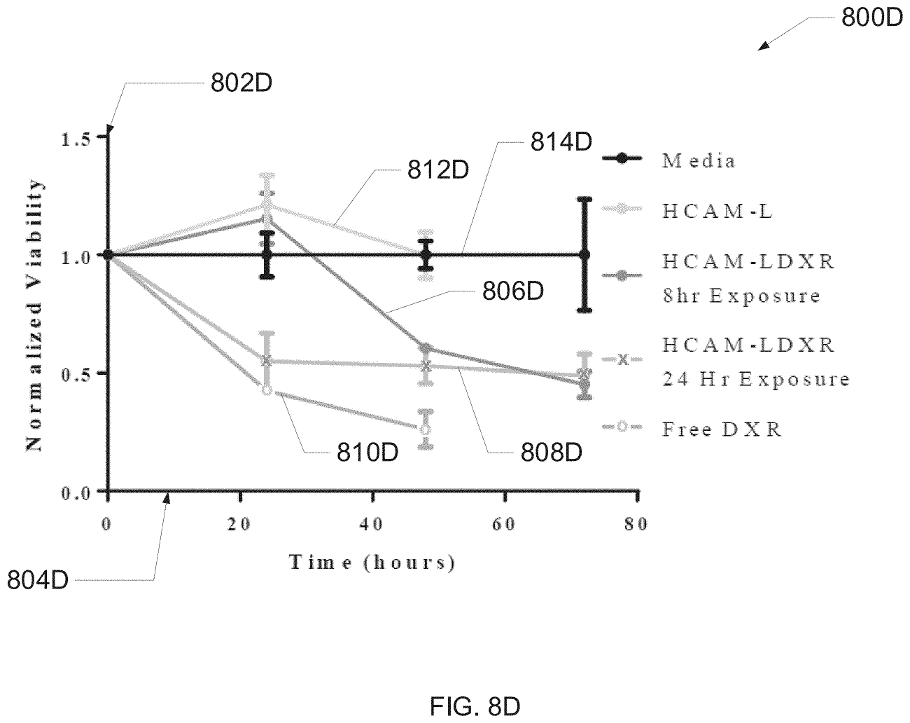

FIG. 8A illustrates a bar graph 800A that relates drug-lipid concentration (D:L) 802A to various agent encapsulation techniques (e.g., conducted for a one hour duration, under a plurality of thermal conditions). The agent encapsulation techniques include: 1) passive encapsulation (“PEC”), wherein encapsulation may be driven solely by a concentration gradient between liposome interiors and surrounding solution (that includes a large volume of active agent); 2) passive equilibration (“PEQ”), wherein a concentration gradient may also be used, but the encapsulation process is further abetted by thermal and temporal conditions intended to increase permeation of active agent into the liposomes. The bar graph 800A further demonstrates relationships between encapsulation of an active agent by liposomes (expressed as D:L) and a temperature at which encapsulation is performed. The bar graph 800A includes a 55° C. PEQ bar 804A, a 37° C. PEQ bar 806A, 4° C. PEQ bar 808A and a PEC bar 810A. Noting that the PEC bar 810A is substantially lower than other bars illustrated, the graph 800A demonstrates that PEQ processes may achieve greater D:L's than PEC processes.

As can be further observed in the graph 800A, the 55° C. PEQ bar 804A presented the highest D:L 802A. Thus, in one or more embodiments, a PEQ process may be best performed at a temperature of 55° C. It should be noted, however, that various lipid components may perform (e.g., encapsulate) dissimilarly under given temperature conditions. Accordingly, the present disclosure provides temperature information in various discrete magnitudes and ranges, but it is also recognized that, while temperature relationships may be consistent across lipid types, temperature magnitudes may vary across lipid types without departing from the spirit of concepts and elements described herein.

Thus, in various embodiments, an effect of temperature on equilibration may be investigated by incubating DXR and liposomes at 4, 37, and 55° C. for one hour. In at least one embodiment (and as is illustrated in FIG. 8A), passive equilibration may increase D:L nearly 200 fold compared to passive encapsulation, and even at low temperatures DXR is associated with the liposomes.

FIG. 8B illustrates a scatter plot 800B that relates D:L 802B to incubation time 804B. In various embodiments, incubation time 804B may refer to a temporal parameter of a passive equilibration process (as described herein). The plot 800B includes a trend line 806B that indicates a maximum value of D:L 802B may be achieved with an incubation time 804B as few as 60 minutes. Thus, the plot 800B and trend line 806B demonstrate that passive equilibration may be suitable for rapid synthesis of active agent-encapsulating, small unilamellar liposomes.

Thus, in one or more embodiments, to optimize a passive encapsulation approach, liposomal samples may be equilibrated for varying durations (up to 8 hours) with DXR. In at one embodiment, at specific time intervals (e.g., such as those illustrated in incubation time 804B) liposomes may purified and an amount of DXR entrapped may be measured (for example, through spectrometry). In one or more embodiments, the liposomes may demonstrate a time dependent increase in DXR encapsulation up to 1 hour (e.g., beyond 1 hour there may be very little change).

FIG. 8C illustrates a scatter plot 800C that relates release of doxorubicin (“DXR”) from liposomes (e.g., that encapsulate the DXR as achieved via passive equilibration at 37° C.) to time 804C, wherein release may be caused by incubating a solution of DXR-encapsulating liposomes at 37° C. in phosphate-buffered saline (“PBS”) with 10% fetal bovine syndrome (“FBS”). As can be observed in the plot 800C, there is a reduction in DXR within the liposomes that results in, at point 806C, 75% of DXR remaining within the liposomes after 48 hours. Thus, the plot 800C indicates that an encapsulated agent (e.g., DXR) may, under proper conditions, escape from encapsulating-liposomes in controlled manner.

Thus, in at least one embodiment, evaluations of release of DXR may be conducted by incubating thermally equilibrated liposomal DXR at 37° C. in PBS with 10% FBS. In one or more embodiments (and as is illustrated in FIG. 8C), results may demonstrate that there is a controlled escape of DXR from liposomes, with 75% (±37%) of the DXR remaining in the liposomes after 48 hours.

FIG. 8D illustrates a scatter plot 800D that relates cancer cell viability 802D to time 804D as observed in a variety of treatments. In various embodiments, to evaluate cancer cell viability in response to homing cell adhesion molecule (HCAM) delivered by various methods. In particular, an evaluated method included integrating, during passive equilibration, DSPE-PEG-MAL within the membranes of liposomes, which when incubated with an antibody (e.g., HCAM), generated potent targeted liposomes. In at least one embodiment, cultures of cancer cells were exposed to a particular HCAM treatment (in some cases, for a specified duration of time) and evaluated following 3 days of incubation. The plot 800D includes: 1) an HCAM-LDRX short exposure trend line 806D, where cancer cells were exposed, for 8 hours, to targeted liposomes incorporating HCAM; 2) an HCAM-LDRX long exposure trend line 808D, where cancer cells were exposed, for 24 hours, to targeted liposomes incorporating HCAM; 3) a free DXR trend line 810D, where cancer cells were exposed to non-encapsulated DXR; 4) an HCAM-L trend line 812D, where cancer cells were exposed to liposomes incorporating HCAM; and 5) a media trend line 814D, where cancer cells were untreated. As can be observed in the plot 800D, trend lines 806D, 808D and 810D demonstrated efficacy in reducing cancer cell viability. In particular, trend lines 808D and 810D demonstrated reduction of cancer cell viability within 24 hours of incubation.

Thus, in one or more embodiments, during equilibration, DSPE-PEG-MAL may be integrated within the membrane, which (when incubated with an antibody, such as HCAM) may generate potent targeted liposomes. In various embodiments, the targeted liposomes may be cytotoxic when introduced to cancer cells in vitro. For example, the targeted liposomes, following introduction, may result in a 52% decrease in cancer cell viability following 3 days of incubation (e.g. wherein all measurements on encapsulated DXR concentrations may be determined by using spectrometry and comparing absorbance values to a standard curve of known concentrations).

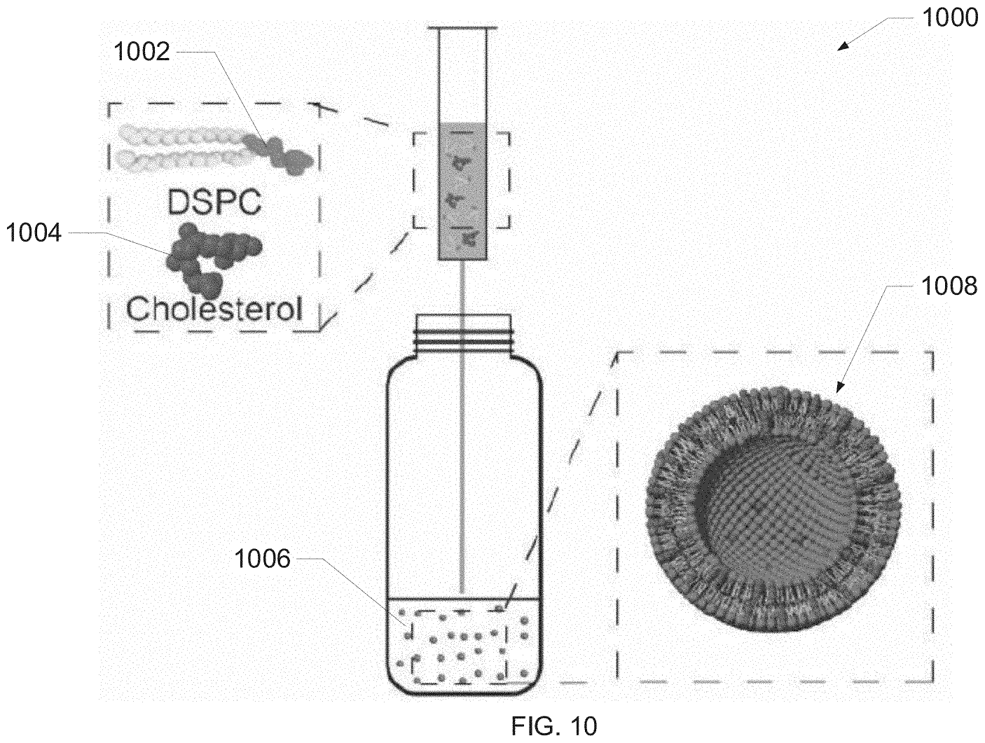

FIG. 10 illustrates an exemplary liposome synthesis technique, according to one embodiment. In one or more embodiments, FIG. 10 illustrates a rapid and scalable synthesis method for synthesizing liposomes. In one or more embodiments, the method may include dissolving membrane components dissolved in isopropyl alcohol and injecting the solution into DI water at 55° C. In one or more embodiments, a resultant phase change causes arrangement of liposome components to form single unilamellar liposomes. In one embodiment, the dilute solution may then be concentrated 20× using filter centrifugation.

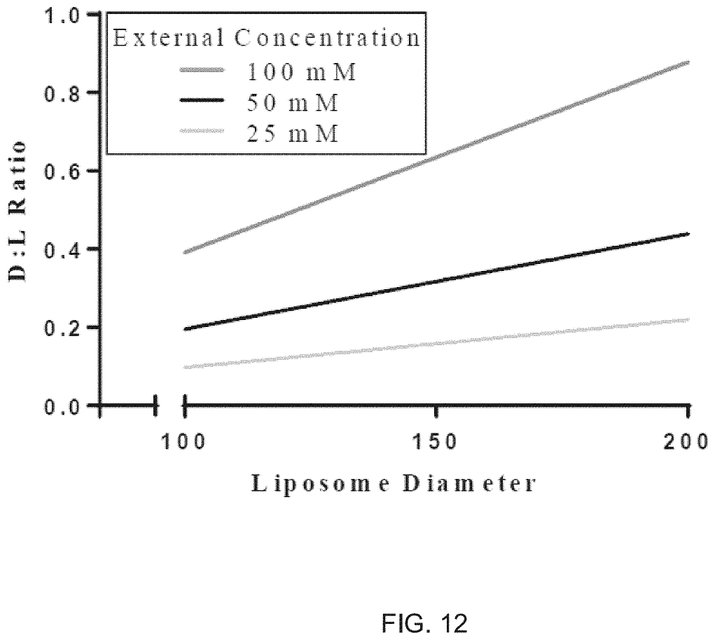

FIG. 12 is a graph illustrating evaluations of exemplary liposome synthesis and encapsulation techniques described herein. FIG. 12 demonstrates that a theoretical D:L ratio of thermal equilibration for hydrophilic molecules may be dependent on liposome diameter and initial drug concentration, where increasing liposome diameter and external concentration results in an increase in liposome potency.

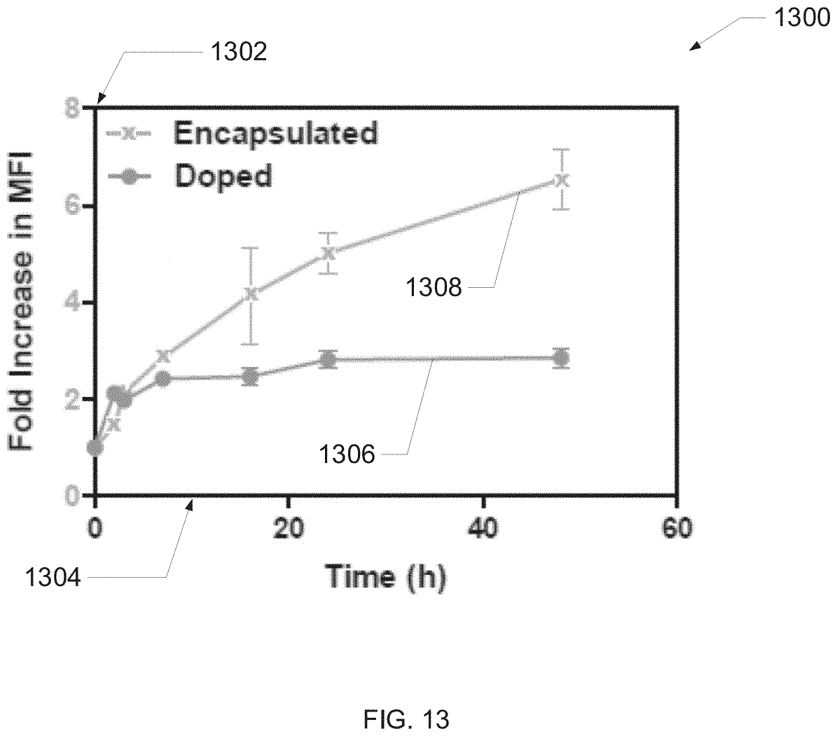

FIG. 13 is a graph illustrating evaluations of exemplary liposome synthesis and encapsulation techniques described herein. In one or more embodiments, an exemplary protocol for evaluating the techniques may include measuring fluorescence intensity of cells treated with either CMFDA-doped and CMFDA-encapsulated liposomes over several hours to measure uptake of CMFDA. In one embodiment, the protocol may further include, but is not limited to, conducting measurements in triplicate on at least 4 independently synthesized and filtered samples (e.g., wherein curves generated from the measurements represent a mean fluorescence intensity (MFI) in respect to time, determined by using a plate reader).

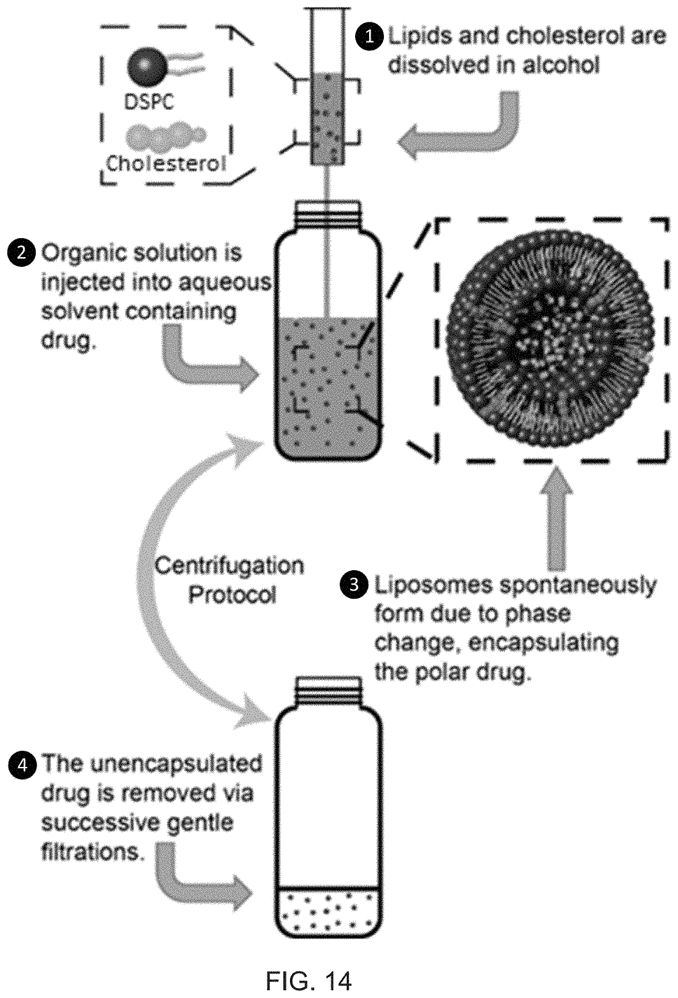

FIG. 14 illustrates an exemplary technique for synthesizing active agent-encapsulating liposomes, according to one embodiment. In various embodiments, lipids may be dissolved in an alcoholic buffer (1) and injected into an aqueous solvent containing the drug of choice (2). In one embodiment, once exposed to the phase change, lipids spontaneously arrange to form SULs, encapsulating the surrounding media in the process (3). In one or more embodiments, non-encapsulated drug may be removed via filter centrifugation (4).

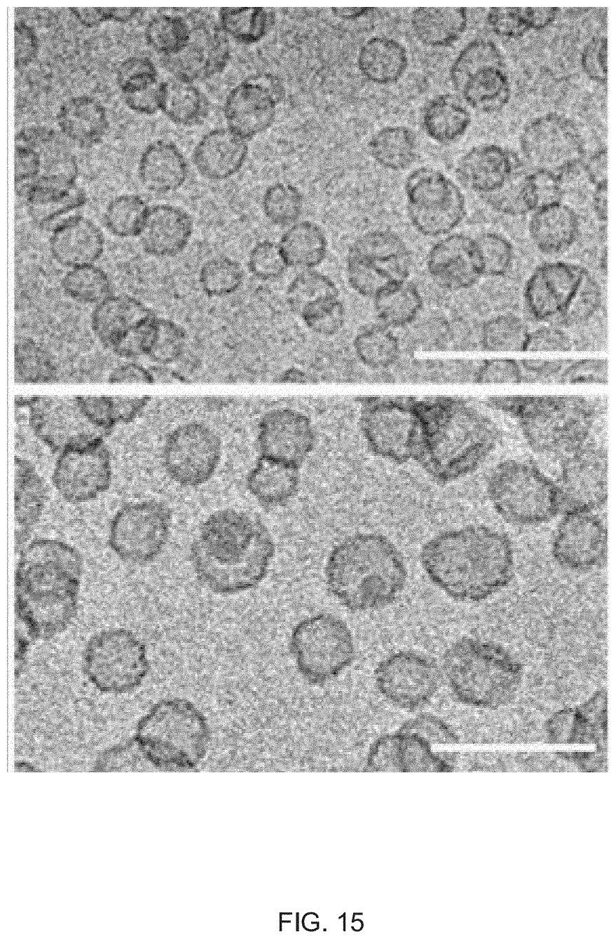

FIG. 15 is an exemplary image set of liposomes synthesized by the present methods, according to one embodiment. In at least one embodiment, FIG. 15 illustrates Cryo-TEM images of liposomes synthesized via an injection method at 12,000× and 25,000× magnification.

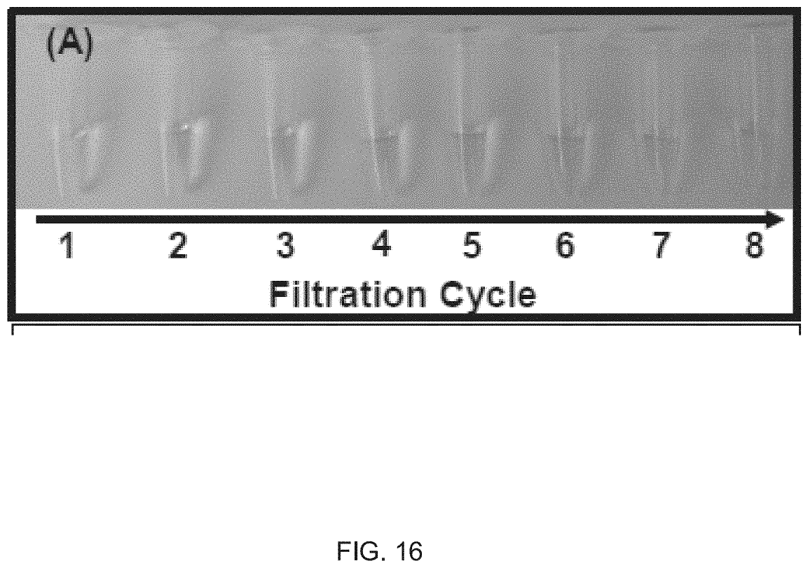

FIG. 16 is an exemplary image illustrating effects of filtration techniques described herein. In various embodiments, FIG. 16 illustrates a change in color of liposomal solutions following each filtration cycle.

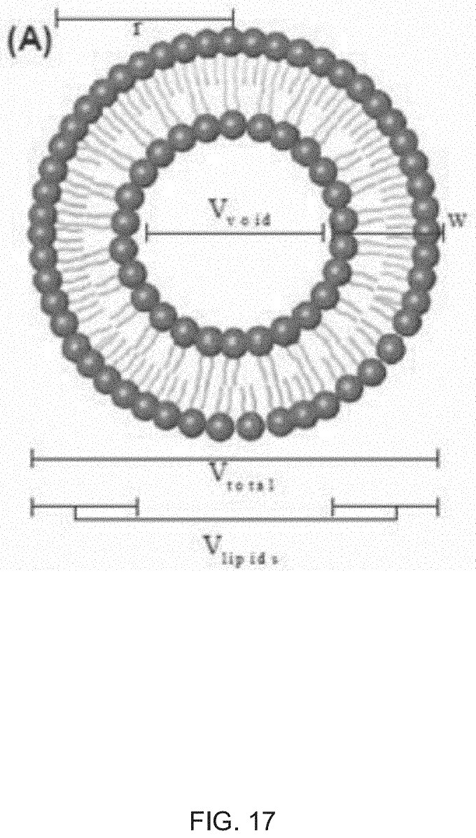

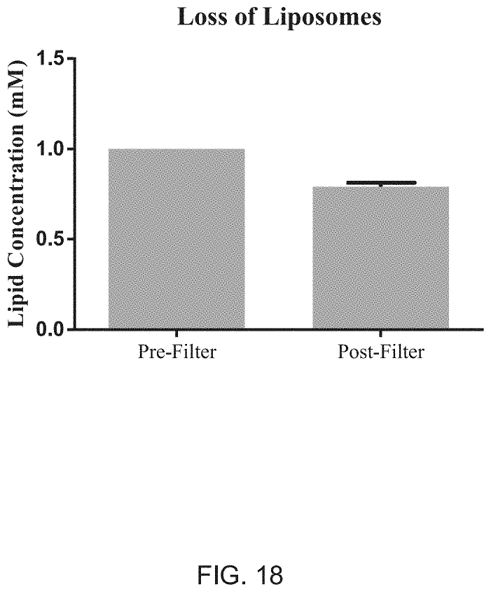

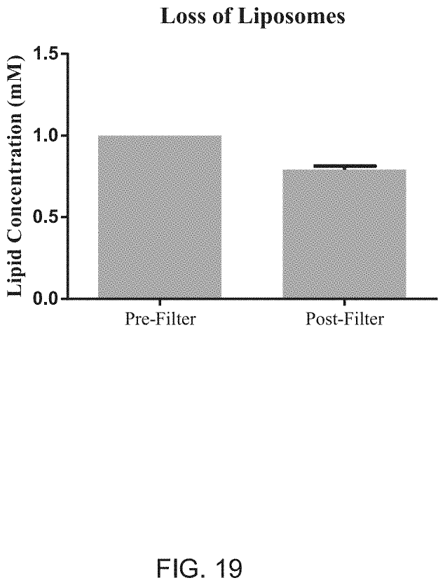

FIG. 17 is a diagram of an exemplary liposome and liposome metrics described herein. In one or more embodiments, FIG. 17 illustrates variables used for estimating a number of liposomes in solution.

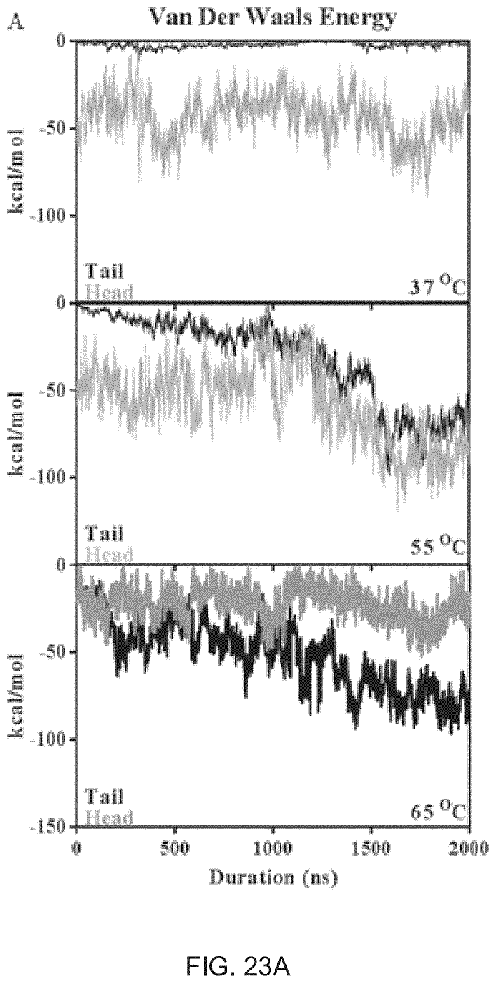

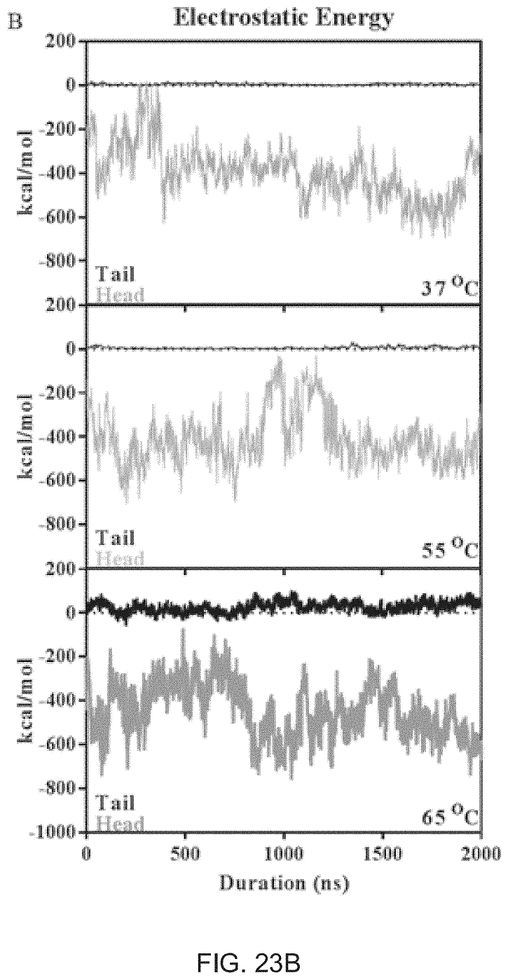

FIGS. 23A-B are graphs illustrating evaluations of membrane dynamics in response to thermal equilibration. In various embodiments, passive equilibration atomic level simulations with DXR may be conducted using NAMD with a CHARMM force field. In at least one embodiment, the simulations may yield analysis of long-range VdW and short range electrostatic forces. In at least one embodiment, at all temperatures, DXR molecules may be attracted to negatively charged lipid heads (e.g., of liposomes in a solution), however; only as the membrane acquires a more liquid-state (≥Tm), may the DXR show any attraction or interaction to lipid tails.

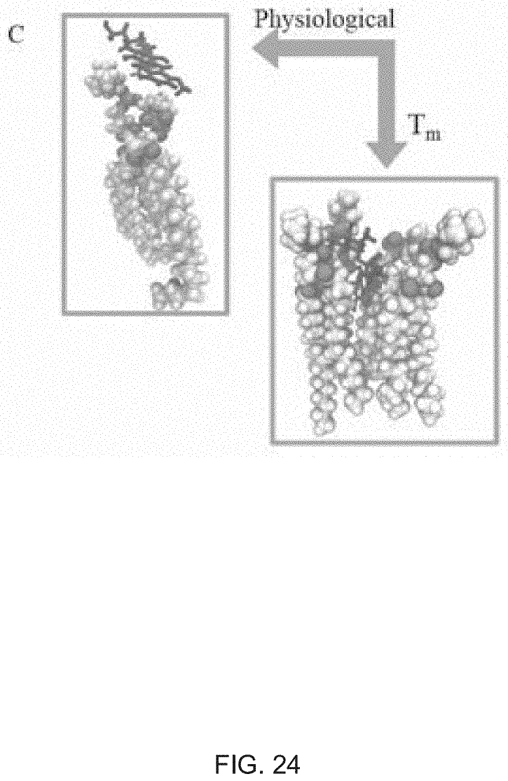

FIG. 24 is an exemplary image of a membrane dynamic simulation and depicts adsorption of DXR to a lipid membrane. In one or more embodiments, FIG. 24 illustrates simulations visualized using VMD. Thus, in at least one embodiment (and as is illustrated in FIG. 24) adsorption of DXR to the membrane may occur below Tm, and, above Tm, the DXR may infiltrate into the membrane.

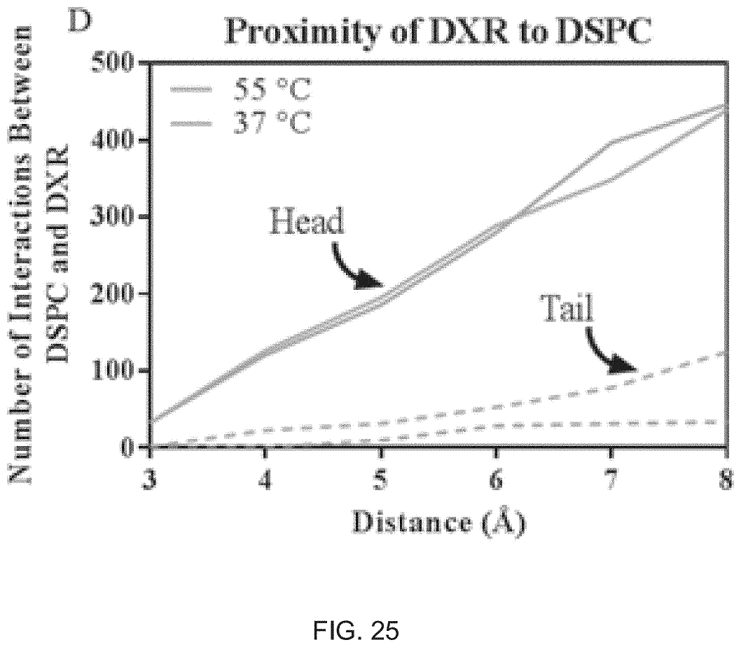

FIG. 25 is a graph illustrating evaluations of exemplary interactions between DSPC and DXR. In one or more embodiments (and as is illustrated in FIG. 25), a large number of DXR atoms may be located within 5-8 Å of DSPC (e.g., in liposomes including each), potentially insinuating a more additive and transient non-covalent interaction.

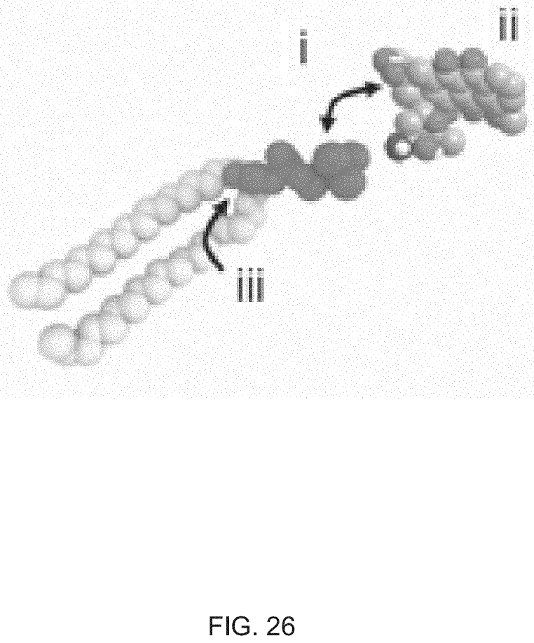

FIG. 26 is an exemplary diagram of a DXR-lipid interaction. In one or more embodiments, stability of the DXR-lipid interactions may be dictated by several factors including hydrogen bonds (i), Brownian motion and diffusion of the DXR (ii), and conformational energy of the tails which allows for diffusion through the membrane (iii).

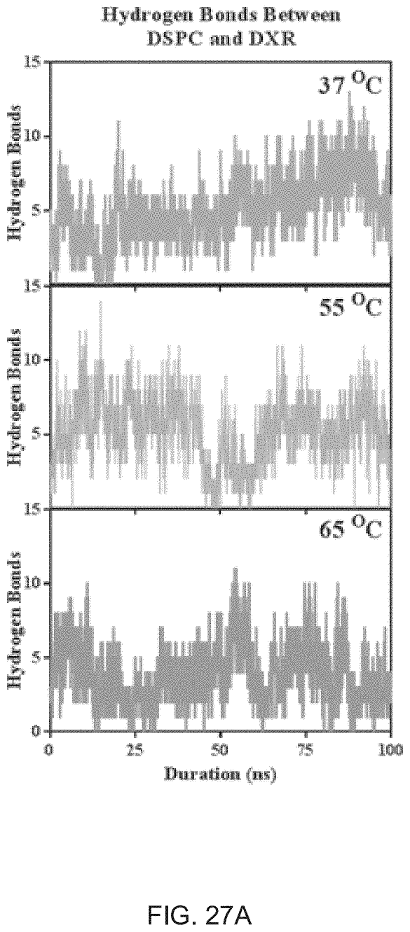

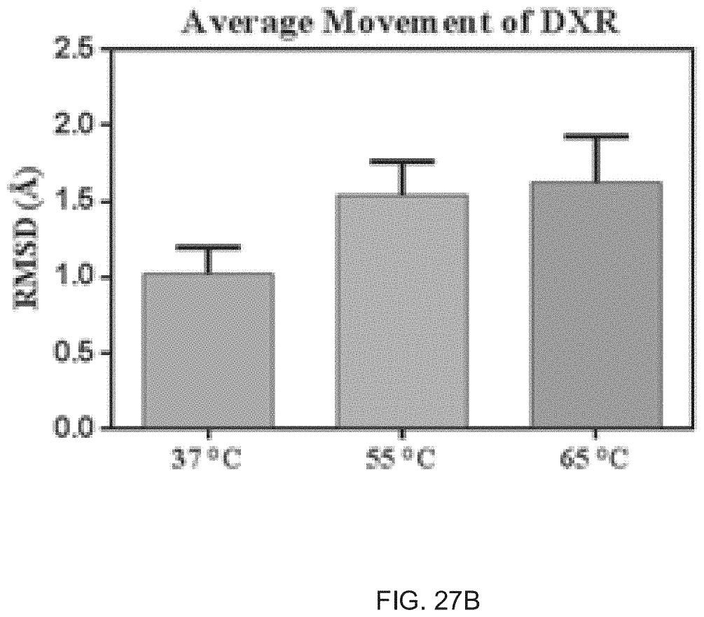

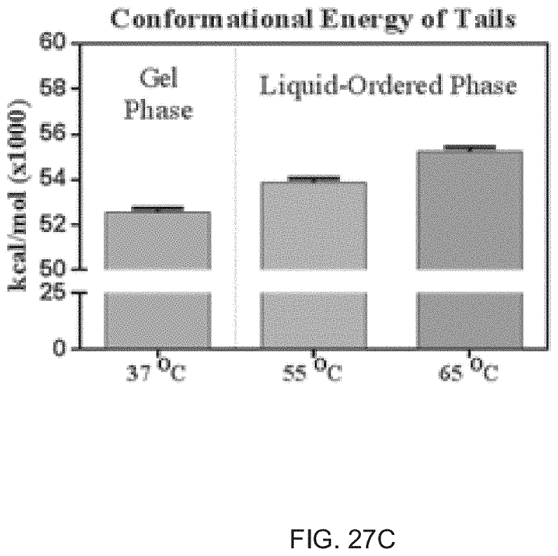

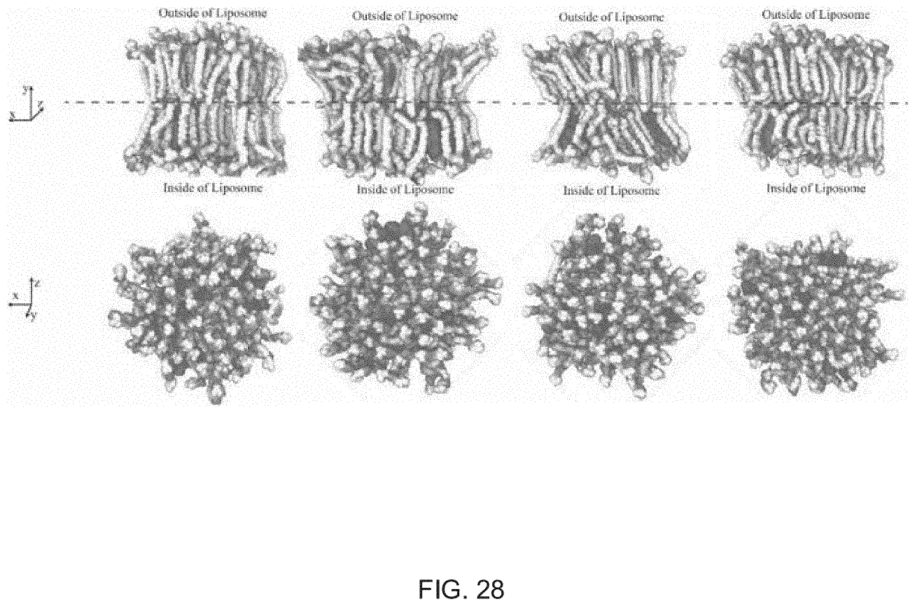

FIGS. 27A-C are graphs illustrating evaluations of exemplary lipids, DXR and DXR-lipid interactions. In at least one embodiment (and as is illustrated in FIG. 27A), DXR may be stabilized both inside and outside a liposome through hydrogen bonding between oxygen and nitrogen atoms on the DXR and phosphates on the DSPC. In various embodiments (and as is illustrated in FIG. 27B), stabilization (along with VdW and electrostatic interactions) may have a profound effect on the movement of the DXR through the liposome. In one or more embodiments (and as is illustrated in FIG. 27C), temperature may affect fluidity of the membrane, and may also play a role in decreasing liposome stability. In at least one embodiment, transitions from 37° C. to 55° C. and from 55° C. to 65° C. may result in an increase in conformational energy of 2.5%, which may be due to non-proportional increase in tail conformational energy as the temperature increases.

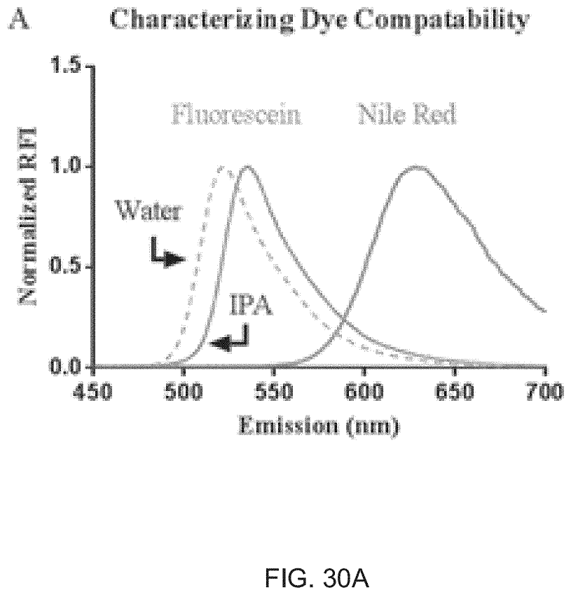

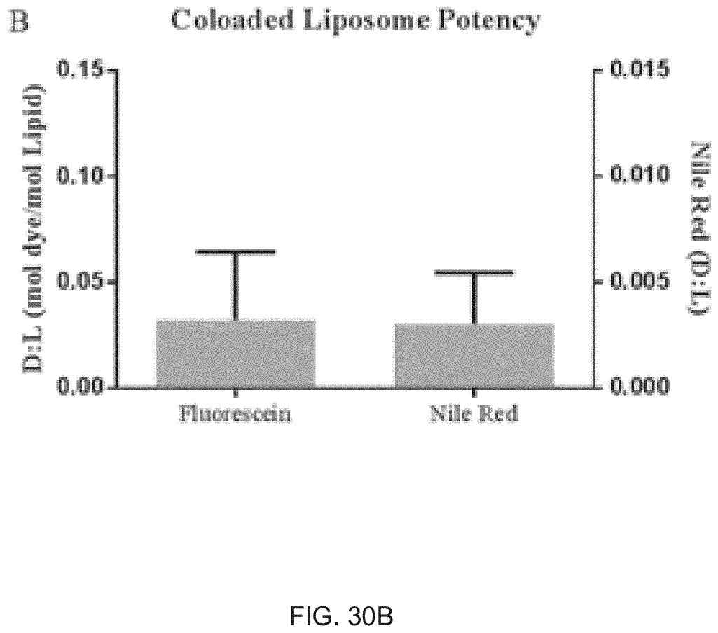

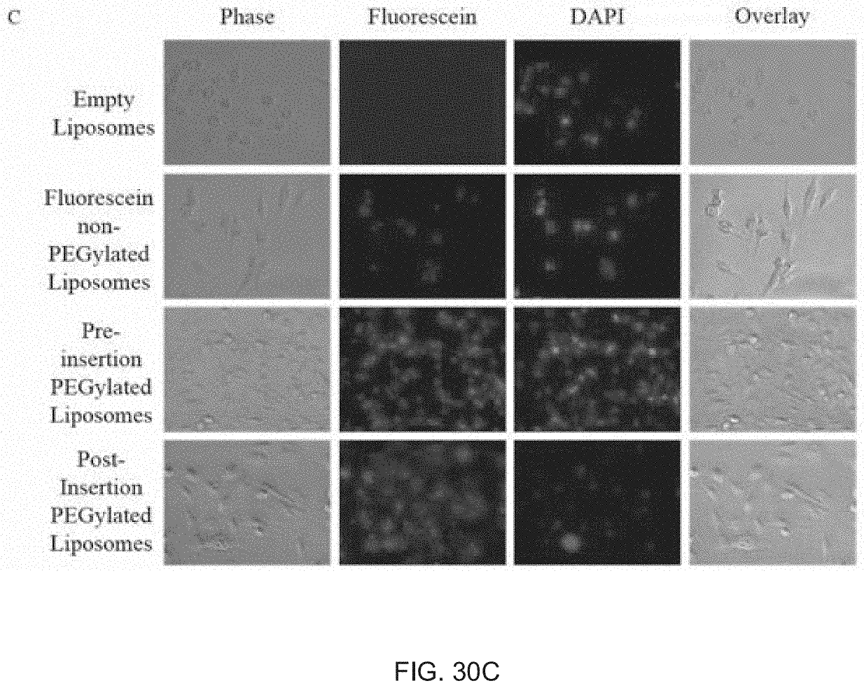

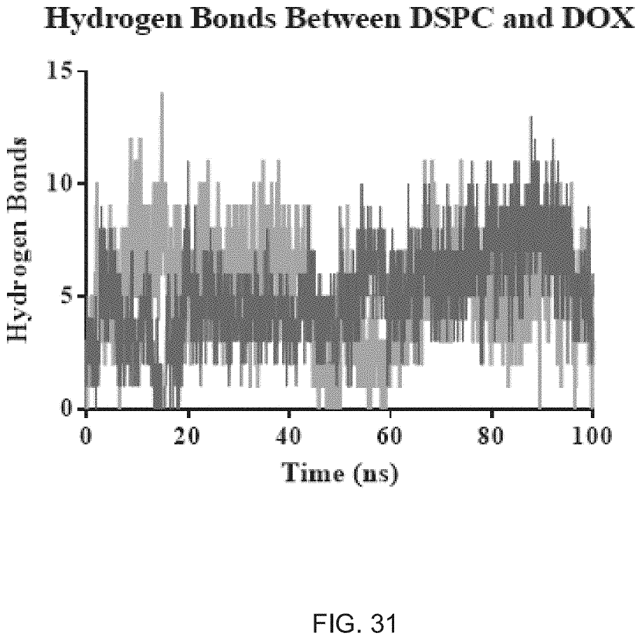

FIGS. 30 A-C are graphs and an exemplary image set illustrating evaluations of thermal equilibration as a universal encapsulation method. In various embodiments, an exemplary protocol for performing evaluations of thermal (e.g., passive) equilibration may include, but is not limited to, using thermal equilibration to load liposomes hydrophilic and lipophilic dyes Fluorescein and Nile Red, respectively. In at least one embodiment, because the dyes differ greatly in fluorescence emission wavelength, the dyes may be easily distinguished in fluorescence imaging techniques.

For example, liposomes may be co-loaded by incubating with a mix of fluorescein and Nile Red. In at least one embodiment, analysis of liposome emission spectra may reveal two peaks indicative of both dyes being sequestered. In one or more embodiments (and as is illustrated in FIG. 30C) liposomes loaded with fluorescein may be targeted to MDA-MB-231 breast cancer cells in vitro using PEG-DSPE-Mal covalently bonded to CD44 as a targeting molecule. In at least one embodiment, the PEGylated lipid may be introduced either during vesiculation or during ET. Accordingly, the cancer cells may be imaged using epifluorescent microscopy with DAPI being used as a counter stain.

### Additional Description of Various Embodiments and Experimental Results

The following section describes one or more experimental tests, and results thereof, performed on one or more embodiments of methods and compositions described herein. The descriptions therein are provided for the purposes of illustrating various elements of the methods and compositions (e.g., as observed in the one or more embodiments). All descriptions, embodiments, and the like are exemplary in nature and place no limitations on any embodiment described, or anticipated, herein.

### Materials

Cholesterol, 1, 2-Distearoyl-sn-glycero-3-phosphocholine (DSPC), and all fluorescent dextrans were purchased from Sigma Aldrich (St. Louis, Mo.). 1,2-distearoyl-sn-glycero-3-phosphoethanolamine-N-[maleimide(polyethylene glycol)-2000] (ammonium salt) (DSPE-PEG-Mal) was purchased from Avanti Polar lipids. Isopropyl Alcohol (IPA, 99% Pure), Ethanol (EtOH, 99% pure), Dimethyl Sulfoxide (DMSO), Trypsin-EDTA, and 5-chloromethylfluorescein (CMFDA) were purchased from Fisher Scientific (Hampton, N.H.). Glass syringes were purchased from Hamilton (Reno, Nev.) and luer lock dispensing needles were purchased from Jensen Global (Santa Barbara, Calif.). Amicon Ultra 100 kDa filter centrifuge tubes were purchased from EMD-Millipore (Billerica, Mass.). A NanoJet syringe pump from Chemyx Inc. (Stafford, Tex.) was used for all experiments. Dulbecco's Modified Eagle Medium (DMEM), Fetal Bovine Serum (FBS), and Penicillin-Streptomycin antibiotic were purchased from VWR (Radnor, Pa.). MDA-MB-231 metastatic breast cancer cell line was acquired from ATCC (Manassas, Va.).

The materials and equipment described herein are exemplary in nature, the present disclosure places no limitations on sources for any material and/or equipment described herein.

### Liposome Synthesis

DSPC and cholesterol were dissolved in alcohol (either isopropyl or ethyl) at a constant 2:1 molar ratio to a final concentration of 10M and 5M, respectively. All experiments were done using these concentrations, unless otherwise noted. The solutions were heated to 50° C. while being shaken at 270 rpm for 15 minutes to ensure adequate mixing of the chemicals. The alcoholic solution was aspirated by a glass syringe and injected into a vial containing distilled water using the syringe pump set to a constant infusion rate (e.g., 50 μL/min). The aqueous solution was constantly vortexed at 600 rpm during infusion and the vortexing continued for 3 minutes following the infusion. All infusions were done at room temperature and 50 μL/minute unless otherwise noted.

### Molecule Encapsulation and Filtration

Liposomes were concentrated 20 fold to a final lipid concentration of 20 mM. Samples were made with 50 μL liposome and 50 μL additive (either distilled water or DMSO) containing the small molecule (DXR, fluorescein, and/or Nile Red). The suspensions were incubated at indicated temperatures and durations. Following this, liposomes were cooled to 4° C. Non-encapsulated DXR was removed from suspension by using a Zeba Spin desalting column with a 7 kDa cutoff, while fluorescent dyes were removed from liposomes using dye removal columns. For targeted liposomes, DSPE-PEG-Mal was dissolved to a concentration of 25 mg/mL in DMSO and added to a final mole ratio of 1% (DSPE/DSPC) along with the additive.

Release of DXR was analyzed following encapsulation via thermal equilibration. Liposomes were synthesized, thermally equilibrated for two hours, and filtered to remove any non-encapsulated molecules. The liposome samples were then diluted 20 fold in PBS with 10% FBS and incubated for up to 48 hours at 37° C. Aliquots were removed at indicated time points and released DXR was filtered from suspension.

Compound concentrations were analyzed using a Thermo Scientific Varioskan Flash multimode reader. DXR concentration was quantified using an excitation of 525 nm and emission of 580 nm. To quantify the concentration of co-loaded samples, liposomes were first analyzed with an excitation of 488 nm and excitation of 512 nm in water to obtain the fluorescence of fluorescein. The samples were then desiccated for several hours under vacuum to remove all fluid and redissolved in IPA. Nile Red levels were then read using an excitation of 550 nm and emission of 625 nm. All intensities were compared emission intensities of their respective standard curves.

### Analyzing Filtration Efficiency and Liposome Morphology

Liposome filtration efficiency was measured collecting the liposome sample following each filtration cycle. The fluorescence of liposomes was measured using a Tecan Infinite 200 Pro (Mannedorf, Switzerland) plate reader and compared to a standard curve made from fluorescent molecules of known concentration. Liposome size and PDI was determined using an N5 Submicron Particle Size Analyzer (Beckman Brea, Calif.).

### Quantifying Phospholipid Concentration

Phospholipid concentration was empirically determined via Stewart's Assay. Briefly, liposomal suspensions were dehydrated at 70° C. in polypropylene microcentrifuge tubes. The lipids were redissolved in 500 μL chloroform, and an equal volume of Stewart's reagent (100 mM FeCl₃ and 400 mM NH₄SCN in DI water) was added and vortexed for 20 seconds. The tubes were centrifuged for 10 minutes at 1000×g to create a phase separation. The chloroform layer was then analyzed for its absorbance at 472 nm and compared to a standard curve to determine the phospholipid concentration. The standard curve was made using a serial dilution of lipids in cholesterol at a 2:1 ratio.

### Cell Culture, Imaging, and Therapeutic Delivery

MDA-MB-231 cells were grown to confluence in DMEM containing 10% FBS and 1% Penicillin Streptomycin. Cells were removed and collected from the culture using Trypsin-EDTA followed by centrifugation. The cells were then reintroduced to a 96 well plate at a concentration of 4000 cells per well and cultured overnight. Non-adhered cells were aspirated and fresh phenol red and serum free media was added prior to liposome introduction. Liposomes were added to the solution at a concentration of 1×10¹⁸ liposomes per well based on the volume estimation (Section 2.3). Fluorescence imaging was done on fixed cells using Zeiss LSM 800 (Oberkochen, Germany) laser scanning confocal microscope.

In one embodiment, MDA-MB-231 cells were grown in DMEM containing 10% FBS and 1× Penicillin-Streptomycin solution. The cells were cultured at 70% confluency and incubated in CMFDA and Hoescht to allow for viability analysis. Following exposure, 8,000 cells per well were transferred to a 96 well plate and incubated undisturbed for two days prior to any experimentation. Liposomal suspensions were UV sterilized prior to introduction to cell cultures. On the second day, the liposomal carriers were introduced to the cultures at the median effective concentration Cell counts were made at each time point by imaging the wells and counting nuclei via ImageJ.

In an alternate embodiment, MDA-MB-231 cells were grown in DMEM containing 10% FBS and 1× Penicillin-Streptomycin solution. The cells were cultured at 70% confluence and plated in a 96 well plate at a density of 5×104 cells/well. The cells were incubated undisturbed for two days prior to any experimentation. Liposomal suspensions were UV sterilized prior to introduction to cell cultures. On the second day, the liposomal carriers containing DXR were introduced to the cultures at the EC-50 of DXR (0.01 μM21). Proliferating cells were quantified and normalized by the control using WST-1 proliferation assay.

### Membrane Simulations

Simulations are performed on a section of the lipid membrane consisting of DSPC and cholesterol held in a 2:1 molar ratio. The membrane section was constructed using Charmm-27 GUI Membrane Builder, and designed to consist of 256 total lipid molecules and 128 cholesterol molecules. The membrane was hydrated using 30 water molecules per lipid. Nanoscale Molecular Dynamics (NAMD) was used to thermally equilibrate the bilayer system for 1 ns at designated temperatures. Production runs were also conducted at designated temperatures with a time step of 1 femtosecond and data collection every 20 femtoseconds. Every simulation was run for at least 1000 ns, yielding at least 5000 data points per temperature. Trajectories were visualized using Visual Molecular Dynamics (VMD). Further information on the simulation setup is located in supplementary information.

### Statistical Analysis

Statistical analysis was conducted using GraphPad Prism 5 (La Jolla, Calif.). For each condition, at least three samples were independently prepared and each sample was analyzed three times. Statistical analysis was performed using either a student's unpaired t-test or a One-Way Analysis of Variance (ANOVA) with a Tukey post-test. Data was deemed statistically significant if p values were less than 0.05. Graphs show the mean and the standard error of mean (SEM) of sample groups.

### Liposome Synthesis and Characterization

Liposomes composed of DSPC and cholesterol were synthesized using the injection method (e.g., as is illustrated in FIG. 14). The organic solution, containing membrane components, was injected into distilled water and the resulting phase change from organic to aqueous caused the spontaneous assembly of liposomal vesicles, encapsulating the surrounding media. Liposomes that were synthesized via this method were visualized using Cryo-EM to ensure unilamellarity (e.g., as is illustrated in FIG. 15). Dynamic light scattering (DLS) was used for subsequent characterizations. The size of liposomes observed through Cryo-EM were slightly smaller than those measured through DLS. This is a phenomenon that has been observed previously and is expected due to both the media in which the particles are observed, and weighting of the size distributions in DLS. DSPC, a synthetic lipid, was used because of its wide range of advantages when compared to other lipids, most notably the higher phase transition temperature (55° C.) due to its longer carbon tail and greater purity. This high transition temperature imparts many favorable characteristics including longer release and degradation kinetics. It has also demonstrated higher encapsulation efficiencies than other similar lipids. However, these characteristics may also complicated the synthesis procedure due to the strong intramolecular forces that resist vesiculation (i.e. rate of membrane closure), leading to polydisperse liposome populations. Several factors, described in the following sections, influence the morphology of liposomes and the process was optimized to rapidly synthesize monodisperse DSPC SUL populations without the need of secondary processing steps.

### Effect of Solvent on Liposome Formation

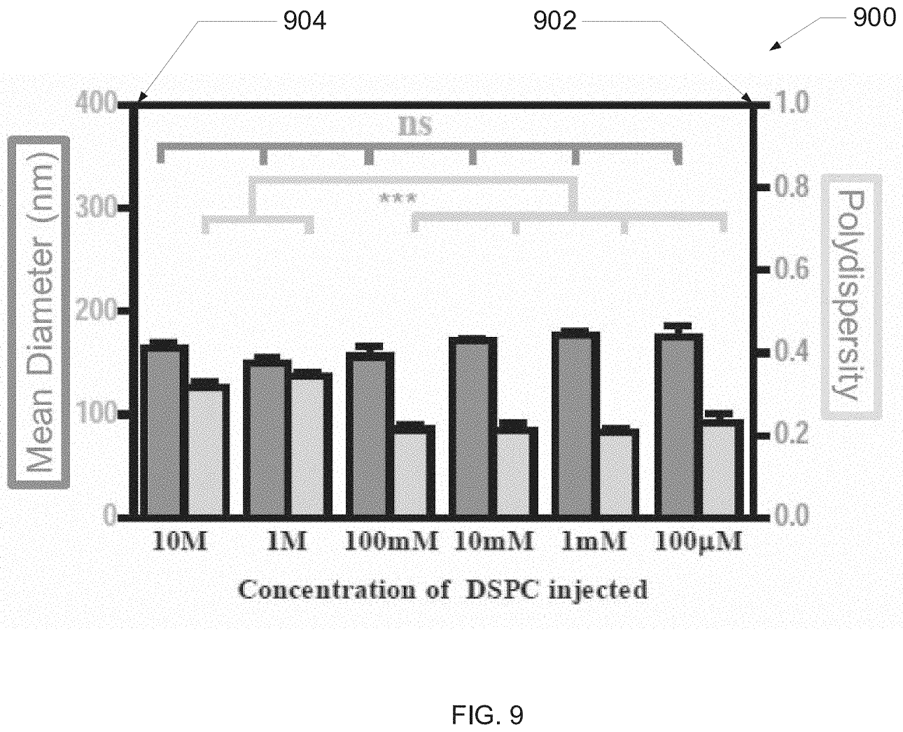

Traditionally, liposomes are synthesized using the injection method by dissolving lipids and cholesterol in EtOH and are injected to a final concentration of 10% (v/v). However, isopropanol (IPA) may be a more suitable solvent for use with other synthetic and natural lipids including Dipalmitoylphosphatidylcholine (DPPC), Dipalmitoylphosphatidylglycerol (DPPG), egg phosphatidyl choline (EPC), and egg phosphatidylglycerol (EPG), as it is a slightly less polar alcohol than EtOH and therefore may stabilize the membrane curvature during vesiculation, while still capable of being miscible in the aqueous buffer. Membrane components were dissolved into either IPA or EtOH with the DSPC:cholesterol ratio being held constant at 2:1. The solution was then delivered to the aqueous buffer to a final alcohol concentration (v/v) of either 5% or 10% (e.g., as is illustrated in FIG. 2). A significant decrease in diameter and PDI (p<0.0001) was observed with higher IPA concentrations resulting in smaller, less disperse particles, reaching a minimum average diameter of 144 nm and PDI of 0.31 with 10% IPA. It is believed that this effect may be due to an increased solubility and therefore, higher stabilization of the lipid bilayer during vesicle formation. Liposomes were synthesized using a 10% final IPA concentration and lipid concentrations ranging from 10 M to 100 μM, while maintaining a constant DSPC:Cholesterol concentration of 2:1 (e.g., as is illustrated in FIG. 9). It was found that as the concentration of lipids in solution decreases from 10 M to 100 mM, the PDI decreases (p<0.0001) from 0.31 to 0.20 with no significant change in mean diameter (p>0.05). No difference was seen in either diameter or polydispersity from 100 mM to 100 μM, indicating that further decreasing lipid concentration may have no effect on liposome characteristics. Thus, by altering the alcohol identity (IPA) and concentration of lipids in the solution, the polydispersity and size of liposomes were effectively decreased without any secondary processing steps such as extrusion or size exclusion filtration.

### Effect of Temperature on Liposome Synthesis

Several mechanisms have been proposed to explain the self-assembly of liposomes during thin film hydration, but the physics underlying the formation of liposomes using injection method is not well understood. It is clear there are several factors that influence the size of liposomes, most notably the vesiculation rate, which can be altered by varying the temperature and the duration for which alcohol solvent stays with the lipids. The precise influence of variations in temperature on liposome size has been minimally explored using microfluidics, and there is limited knowledge available regarding its effects on liposomes formed using the injection method. To further investigate the influence of temperature during liposome formation, the temperature of aqueous buffer was incrementally increased to the liposomal phase transition temperature (55° C.). Despite seeing a 33% decrease in PDI when reducing lipid concentrations from 10 M to 100 mM at room temperature, there was no reduction in PDI for solutions made with lower lipid concentrations at high temperatures (data not shown). Therefore, to increase the total concentration of liposomes in solution, a 10 M solution of lipids was used. A significant (p<0.0001) change in both size and PDI was observed when temperature increased from 4° C. to 55° C. (e.g., as is illustrated in FIG. 3A). At relatively lower temperatures below the transition temperature (<37° C.), the liposomes were highly polydisperse (0.75-0.36) with mean diameters near 200 nm. This is likely due to an increase in membrane rigidity, which resists bending of the bilayer and thus slowed the closure speeds of liposomes. As temperatures neared the phase transition of DSPC (50-55° C.), the lipids convert from a solid gel phase to a disordered liquid phase with increased vesiculation rates. This phase change decreased the rigidity of the lipid membrane, thereby increasing the closure rates and directly affecting liposome size and PDI. Near the transition temperature, liposome diameter decreased to 80 nm in diameter and 0.13 in PDI, without the aid of any secondary post processing steps. These results indicated that temperature is a factor that can be exploited to control liposome size and polydispersity, and by increasing the temperature of aqueous medium in which liposomes are produced to near transition temperature, synthesis of populations of monodisperse DSPC SULs less than 100 nm in diameter was possible.

### Effect of Flow Rate on Liposome Synthesis

Production of liposomes in an economical, efficient, and scaled manner would require large amounts of materials to undergo the synthesis process. Increasing flow rate of the alcoholic solution is a simple way to drastically increase the overall throughput. However, little is known about the effect of shear stress and turbidity on the formation of liposomes. Using microfluidic devices with hydrodynamic focusing, researchers have postulated that the size and PDI of liposomes can be fine-tuned by controlling shear of laminar flows. It is believed by the present inventors that all shear imparted on the lipids within the syringe and at the aqueous:organic interface are negligible compared to the convective forces within the vortexing aqueous buffer. By increasing the infusion rate, the throughput of the disclosed synthesis protocol can be enhanced. A range of flow rates of alcoholic solution into the aqueous buffer was tested and found no significant difference (p>0.05) from 25 μL/minute to 400 μL/minute (e.g., as is illustrated in FIG. 3B) However, above this range, the liposome size increased to 133 and 212 nm for flow rates of 800 μL/minute and 1200 μL/minute respectively. Similarly, the PDI slightly increased to 0.3 and 0.38 (p<0.0001) respectively. This change was attributed to an increase in lipid concentration at the needle tip when flow rates are higher, and not due to any fluctuations in shear or turbidity. Additionally, no significant difference was found when decreasing the gauge of needle, further validating that the shear rate during injection does not affect liposome size or distribution.

### Filtration and Concentration of Drug Loaded Liposomes

A noted drawback of the injection method is that passive encapsulation techniques have a low efficiency for capturing drug molecules within liposomes. Unlike active encapsulation techniques where sequestration is driven via transmembrane gradients, passive techniques sample the surrounding medium, and close around the aqueous drugs during liposome self-assembly. This leads to a large concentration of non-encapsulated drug in suspension that needs to be removed prior to usage. Most studies encapsulate small drugs such as doxorubicin, and remove the non-encapsulated molecules using ultracentrifugation or dialysis. However, not much is known about the encapsulation and removal of larger molecule drugs (>1000 Da), which may be desirable in cases such as long term insulin release, adoptive cell therapy, or gene therapy. Regardless, the purification techniques are lengthy and the limited availability of necessary instrumentation can be prohibitive and especially limits the point-of-care use. Chromatography and dialysis, while capable of purifying liposomes, cannot adequately concentrate the liposomes, creating dilute solutions that need further post processing techniques (e.g. lyophilization or speedvac). Ultracentrifugation has also been shown as an effective purification method, however it necessitates multiple cycles of centrifugation at >100 k×g for several hours and can be damaging to fragile liposomes. Additionally, a large number of liposomes are lost after each cycle due to the low sedimentation coefficient associated with SULs. To address these limitations, filter centrifuge tubes containing pores large enough to ensure sieving of large molecules, yet small enough to inhibit loss of liposomes in the colloidal solution were used to rinse the synthesized liposomes.

Liposomes were synthesized using an optimized protocol (10M, 55° C. and 400 μL/minute). Fluorescein molecules of varying molecular weights were then doped into the colloid and filter-centrifuged at 6000×g for 10 minutes (for reference, standard benchtop centrifuges can achieve rotation speeds of 15,000×g). The fluorescence of the liposome solution was analyzed using a plate reader following each filtration cycle, and used as a measure of concentration in the solution (e.g., as is illustrated in FIG. 4A and FIG. 16). In each case, 6 filtration (rinse) cycles result in a net removal of 99% of the non-encapsulated drugs, while 8 filtration cycles result in a net removal of 99.9% of the non-encapsulated drugs. DLS was used to measure the size and polydispersity of liposomes before and after filtration. A slight change in diameter and polydispersity was observed (e.g., as is illustrated in FIG. 4C), with an increase in average diameter by 15 nm and decrease in PDI by 0.08 (n=12, p<0.0001), indicating that no agglomeration occurred as a result of centrifugation. This slight increase in diameter was attributed to the filtration of small insoluble particulates in the media and not to loss of any SULs. The rotational speed of the centrifugation approach is nearly 20× slower than typical ultracentrifugation rotational speeds. Furthermore, the complete synthesis and filtration of monodisperse SULs took less than three hours, which is remarkably faster than commonly employed protocols such as thin film deposition with ultracentrifugation that can normally take several days for the entire process. Finally, the sensitivity of DLS allowed for simplistic testing of sterility of nanoparticle solutions, with the presence of multiple peaks (≥1 micron) indicating possible contaminations. However, only single peaks in DLS were observed, demonstrating the ability of disclosed methods to maintain sterility. Therefore, using sequential rounds of filter centrifugation, the concentration of non-encapsulated molecules within the solution were effectively depleted while maintaining the integrity of synthesized liposomes.

### Synthesizing Liposomes for Drug Delivery

The primary use of liposomes is delivery of therapeutics or active agents that may either be sensitive to clearance by the reticuloendothelial system, or may be cytotoxic to the host. Therefore, it is optimal that non-encapsulated molecules be removed from the solution while maintaining the bioactivity of the encapsulated drugs. To confirm that sequestered liposomal molecules are released following filtration, cell tracker CMFDA was encapsulated using the previously optimized approach. This molecule allows for convenient analysis as it becomes fluorescent only upon entering the cell and being hydrolyzed by esterases. In order to deliver identical concentrations of CMFDA across samples, the number of liposomes in suspension (Nₜₒₜal) was estimated based on the volume method:

$${N}_{total} = \frac{{V}_{phospholipids}}{{V}_{lipids}} \qquad ( Equation 1 )$$

where V_{phospholipids} is the volume of the phospholipids in the alcohol solution and V_{lipids} is the volume of the lipids within the shell of the liposome. V_{phospholipids} can be found as the quotient of the lipid mass in the alcohol (M_{lipid}, here 7.9015 mg mL⁻¹) divided by the density of the lipid (p, here 1.03 g·mL⁻¹ at 55° C.):

$${V}_{phospholipids} = \frac{{M}_{lipids}}{ρ} \qquad ( Equation 2 )$$

V_{lipids} can be estimated by subtracting the void volume (V_{void}) of the liposome from the total volume (Vₜₒₜₐₗ) of the liposome:

$$\begin{matrix}{V}_{lipds} = {V}_{total} - {V}_{void} & ( Equation 3 ) \\ {V}_{total} = {}_{3}^{4} π {r}^{3} & ( Equation 4 ) \\ {V}_{void} = {{}_{3}^{4} π ( r - w )}^{3} & ( Equation 5 )\end{matrix}$$

where r is the radius (determined by DLS), and w is the membrane thickness (determined to be 3 nm by atomic force microscopy). In at least one embodiment, the liposome parameters of the above equations (e.g., Equations 1-5) may be illustrated in FIG. 17.

As a control, blank liposomes were also synthesized and CMFDA was doped into the colloidal solution. All samples were filtered to remove non-encapsulated molecules, therefore depleting the control of all CMFDA. MDA-MB-231 cells were cultured and exposed to identical concentrations of liposomes (1×10¹⁸ liposomes/well). The fluorescence of the cells was recorded at regular intervals for 48 hours (e.g., as is illustrated in FIG. 13).

Liposomes with CMFDA encapsulated in the core progressively increased the mean fluorescence intensity (MFI) of the solution 6.5 fold within 48 hours (e.g., illustrated in the trend line 1308 of FIG. 13). Although it was assumed that the control samples would have no increase in fluorescence, there was a 2.9 fold increase in the MFI. This increase was attributed to advantageous binding of hydrolyzed CMFDA molecules to the shell of the liposomes and not to remaining non-encapsulated molecules in the solution, as the increase in fluorescence occurred several hours after introduction of the liposomes (CMFDA enters the cell and becomes fluorescent within 30 minutes) and no increase was observed after 8 hours. Following the 48 hour incubation, cells were fixed using paraformaldehyde and imaged using a confocal microscope (e.g., as is illustrated in FIG. 5). Green fluorescence was present in cells that were incubated with filtered CMFDA encapsulated liposomes, but not with the cells that were incubated with liposomes doped with CMFDA and subsequently filtered. The disparity in intensity between cells incubated with CMFDA loaded liposomes and those incubated with control samples validates that drug loaded liposomes synthesized by the simplified approach proposed here can be used as potent drug carriers for therapeutic delivery.

During the process of drug development, extensive pre-clinical studies are performed to help understand the molecular level interactions between the therapeutic and the patient. However, even in the best scenarios, the translation from a pre-clinical to a clinical study is time consuming, expensive, and often fails due to low efficacy, specificity, or therapeutic index. This barrier has led to a decline in novel pharmaceutical agents which have entered the market, and an increase in efforts to develop systems which increase the control over drug delivery. DDSs have been engineered to alter the solubility, biodistribution, and targeting capabilities of the drug while eliminating many adverse effects that occur as a result of off-targeting. However, development and implementation of novel DDS's require tedious protocols and equipment not readily available in most clinical or laboratory settings.

Disclosed herein are method and compositions to rapidly produce a monodisperse population of drug loaded SULs using benchtop equipment commonly found in most laboratories. By altering the synthesis conditions, small unilamellar DSPC liposomes in the range of 100 nm can be reliably produced. Despite the higher cost and challenges associated with synthesizing SULs from gel phase phospholipids like DSPC, these synthetic and semisynthetic phospholipids offer several advantages compared to natural phospholipids. Aside from the previously mentioned advantages of higher encapsulation efficiencies and transition temperature achievable with DSPC phospholipids, many of the PEGylated phospholipids used for targeting and stealth capabilities are synthesized from DSPC precursors, facilitating insertion of these molecules into the bilayer. Furthermore, liposomal formulations prepared with DSPC exhibit longer plasma circulation lifetimes.

Following synthesis of drug-loaded SULs (active agent comprising liposomes), it was necessary to filter out non-encapsulated molecules from the solution. Methods disclosed herein were capable of filtering molecules up to 20 kDa with 8 ten-minute centrifugation cycles, which efficiently increased the purity of the samples up to 99.9%. This also illustrated the necessity for multiple filtration cycles, as many established protocols only utilize a single filtration or ultracentrifugation cycle without a reported control to ensure complete sieving. The filtration inserts would lose their sieving ability following the second consecutive use. This is likely due to the high amounts of excess dye resulting in concentration polarization across the filtration membrane. However, sieving efficiency was restored by washing the filters twice with 70% IPA and once with deionized water to remove any residual IPA. By following this approach, the sieving efficiency of the units was restored. Each unit was used for at least ten wash cycles (20 total filtrations).

Methods and compositions disclosed herein were used to deliver a fluorescent molecule, CMFDA, which is cell permeable in its inactive form, and becomes fluorescent only after being activated within the cytoplasm of the cell. Methods and compositions disclosed herein produced sterile populations of liposomes containing the CMFDA molecule within three hours. This method was remarkably faster than the current methods (reduces processing time from days to a few hours), while utilizing commonly available benchtop equipment. Methods and compositions disclosed herein will reduce the common limitations in DDS research and lead to further translational innovations and novel clinical discoveries for therapeutic delivery.

### Synthesis Approach and Maximum Equilibration Calculation

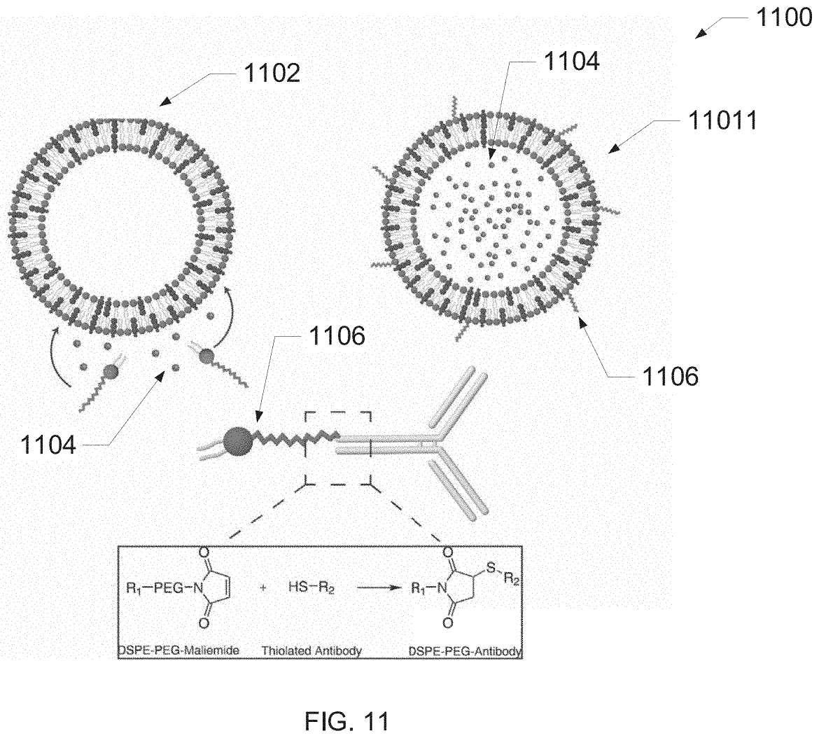

The disclosed technique relies on the incubation of concentrated liposomal solutions, where the ratio of lipid:drug can be optimized to enhance potency of the vehicle without using large amounts of the therapeutic compound. Liposomal solutions are first synthesized and then concentrated using centrifugal filters (e.g., as is illustrated in FIG. 10). Generally, centrifugal filtration is much more advantageous than the commonly used ultracentrifugation (e.g., as is illustrated in FIGS. 6A-B). In addition to a smaller footprint, filter centrifugation retained more liposomes than ultracentrifugation, as determined by Stewart's Assay. The drug and liposomes are allowed to incubate for enough time to eventually equilibrate. Overtime, the drug molecules diffuse through the membrane and into the liposome with the concentration of the therapeutic eventually converging to equal the external concentration (e.g., as is illustrated in FIG. 11). This approach greatly simplifies the tedious encapsulation procedure and does not require the specialized ion or pH gradients that can preclude certain drugs.

Typically, the ability of an approach to sequester small molecules is defined by the encapsulation efficiency (% EE),

$$\% EE = \frac{{C}_{e}}{{C}_{t}} \qquad ( Equation 6 )$$

Where, Cₑ is the moles of drug inside the liposome and Cₜ is the total moles of drug used. This metric is useful as a general description of the loading method, and in remote loading where the drugs are sequestered through ion exchange mechanisms (and the moles of ions are in excess), however it is irrelevant in passive approaches where % EE can be increased solely by increasing the number of liposomes synthesized. A more useful metric that has also been used by researchers is the drug:lipid ratio (D:L), which is defined as:

$$D \text{:} L = \frac{{C}_{e}}{{C}_{l}} \qquad ( Equation 7 )$$

Where, Cₗ is the moles of lipid in the sample. This calculation better explains the therapeutic index of the delivery vehicle, and more accurately depicts the potency of the drug. However, in the disclosed approach D:L is limited to the equilibrium state, where the Cₑ will eventually converge to Cₜ. To better describe the equilibration approach, evaluate the maximum equilibration (Cmax), where the number of molecules found within the core of all liposomes in solution will be proportional to the number of drug molecules found within the surrounding media, with their constant of proportionality (a) being the ratio of the respective volumes (V_{Excluded} for internal pore and V_{Total} for total volume).

$$α = \frac{{V}_{Excluded}}{{V}_{Total}} \\ {C}_{T} \geq α \cdot {C}_{E} \qquad ( Equation 8 )$$

V_{Excluded} can be estimated by determining the inner volume of one liposome, and extrapolating that quantity out for all liposomes in the sample. Furthermore, if the drug is lipophilic, V_{Excluded} can be substituted for the volume of the lipid membrane.

A predominant benefit to liposomal based nanocarriers is their ability to encapsulate pharmaceuticals with varying lipophilicities. However, choosing an efficient approach for encapsulating target compounds can be difficult. Typically the approach is chosen based on the molecules partition coefficient (logₚ), an intrinsic value used to describe a compound's solubility. A benefit in using passive encapsulation is that it can be used to capture small molecules despite their logₚ, where lipophilic molecules (logₚ>0) are dissolved in the organic phase and hydrophilic molecules (logₚ<0) are dissolved in the aqueous phase prior to synthesis. Using the disclosed model, the D:L ratio can be predicted as a function of concentration and liposome size (e.g., as is illustrated in FIG. 12). Assuming that the density of the lipid membrane doesn't change as liposome size changes, the maximum equilibration of lipophilic drugs is solely dependent on Cₜ.

### Stability of Liposomes During Equilibration

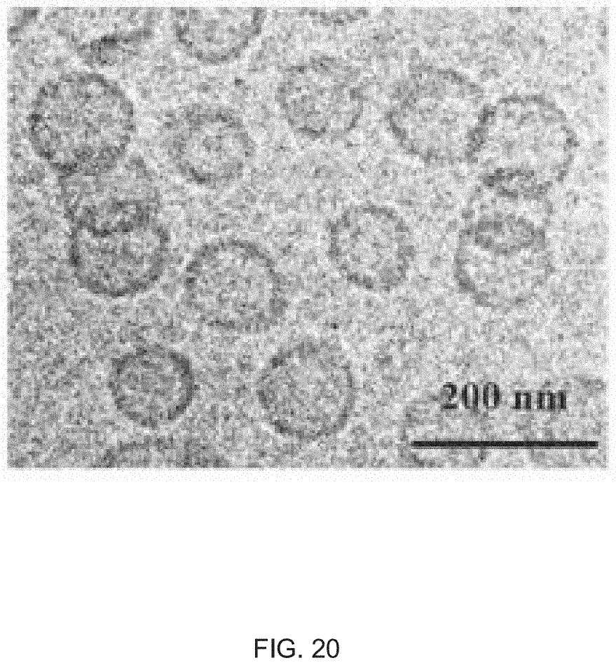

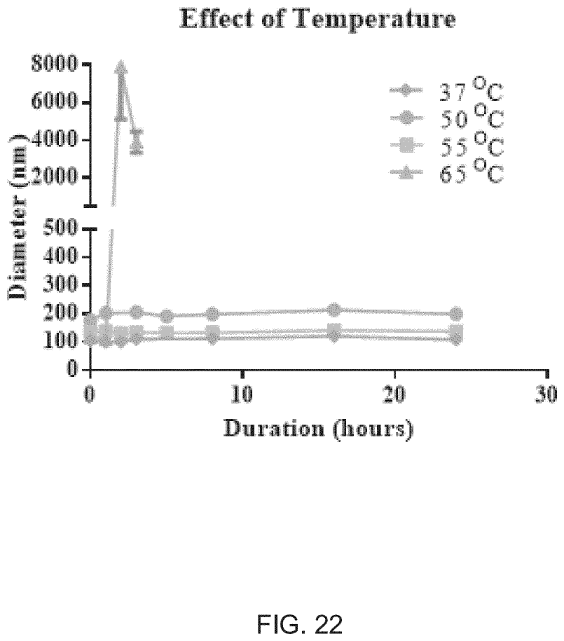

In order to diffuse into through the membrane, small molecules must migrate through both hydrophilic and hydrophobic regions. Below Tₘ, the lipid membrane is in an ordered gel state, where the hydrophobic tails are very stable and show little random motion. However, beyond Tₘ, the membrane converts to a disordered liquid-state, enhancing fluidity and permeability. Initially, liposomes synthesized and concentrated are near 117 nm with polydispersities of around 0.1 (e.g., as is illustrated in FIG. 20). These liposomes are generally stable for up to 4 weeks when kept refrigerated at 4° C. Stability of the liposomes were analyzed at varying temperatures: physiological (37° C.), below Tₘ (50° C.), Tₘ (55° C.), and above Tₘ (65° C.). Liposomes were stable for up to 24 hours at temperatures≤Tₘ (e.g., as is illustrated in FIG. 22). However, above the transition temperature, liposomes were only stable for one hour, as seen in the abrupt change in mean diameter. Precipitates were seen forming in samples incubated at 65° C. for more than 8 hours.

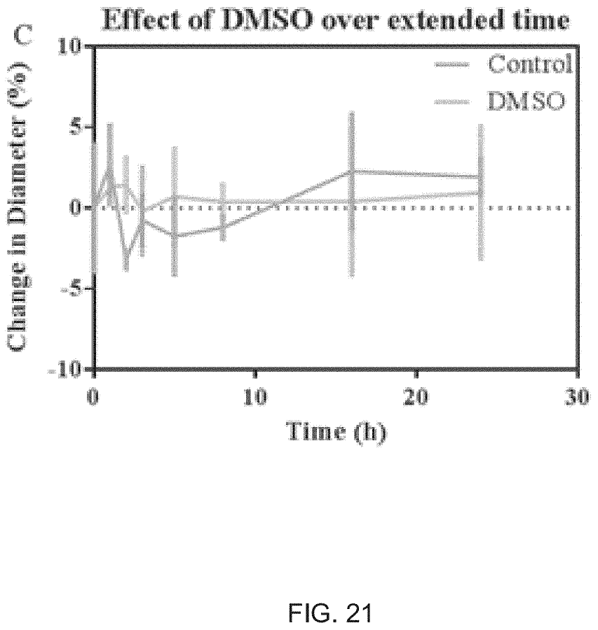

In order to solubilize and encapsulate lipophilic molecules, an organic, water miscible solvent must be introduced to the liposome suspensions. However, the concentrations and identity of the organic must be carefully chosen as to efficiently solubilize the hydrophobic therapeutic, without disrupting the delicate liposomal membrane. Alcohols such as isopropanol and ethanol offer convenient methods of increasing solubility of many drugs, however these solvents can also deactivate or precipitate some drugs. DMSO is an alternative that is widely used to enhance the solubility of nonpolar chemicals for drug delivery. All three chemicals were incubated with concentrated liposomal slurries at 55° C. and their size change analyzed. 10% DMSO had the least effect on liposome size (e.g., as is illustrated in FIG. 7), with an average shift from 104 nm (+0.35 nm) to 107 nm (+0.55 nm). Liposomes immediately precipitated out of solution when introduced to alcohol concentrations of greater than 40%. Samples that include DMSO were stable for up to 24 hours at 55° C. (e.g., as is illustrated in FIG. 21). However, similarly to the samples without DMSO, their stability rapidly diminishes when above transition temperature.

Doxorubicin was chosen as a model drug as it is widely used in its liposomal form (Doxil®) as a first line chemotherapeutic for a variety of cancers (e.g., breast, lung, ovarian, etc.). DXR, is relatively hydrophilic (logₚ-0.631) with an excitation and emission spectra of 500 nm/600 nm. Liposomes were equilibrated with Doxorubin, with initial drug concentration kept constant at 1:1.72. The liposomes readily sequestered DXR with a clear exponential trend to increase as temperature increases up to 1 hour (e.g., as is illustrated in FIG. 8A). Liposome slurries were incubated with DXR at varying temperatures ranging from 4 to 55° C. with DXR, with initial lipid to drug concentrations again held constant at 1:1.72. The disclosed approach was compared to another commonly used “universal” approach, passive encapsulation. Equilibration at Tₘ produced nearly 200× times higher D:L ratios than using the injection form of passive encapsulation (0.000239 and 0.045%, respectively). There was no significant difference between equilibrated samples below Tₘ (n=3, p=0.5396), however equilibration at 55° C. increased the amount of encapsulated DXR fourfold.

### Delivery of Chemotherapeutics from Equilibrated Liposomes

One advantage liposomes have over other forms of nanodelivery systems is the ease in which they can be customized and targeted to specific cell types. Typically, this is done by adding a functionalized PEGylated lipid to the organic solvent during liposome synthesis. Upon hydration, the PEGylated lipid is incorporated on both sides of the membrane bilayer. This approach removes precious space from the internal compartment of the liposome, will also increasing the opportunity for therapeutics to interact with functional groups in the core. Alternatively, liposomes can be incubated with PEGylated lipids at the Tₘ. The PEGylated lipids insert themselves into the membrane, allowing for future customization and functionalization, which is how the disclosed approach created targeted potent liposomes using the equilibration method.

Liposomes were incubated with DXR (17.2 mM) and DSPE-PEG-MAL (0.1 mol %) for 1 hour. The suspension was then purified using spin columns and incubated overnight with CD44 antibodies as described in the methods. Targeted liposomes were introduced to cultures of MDA-MB-231 cells and allowed to interact for 8 hours or 24 hours prior to being washed. As a negative control, liposomes targeted liposomes containing no DXR were also allowed to attach for 24 hours, while free DXR at the ED50 was used (0.58 μM) as a positive control. Viability was analyzed over 72 hours using WST-1 as an indicator of proliferation and normalized to a media only control (e.g., as is illustrated in FIG. 8D). Cells showed a time dependent decrease in proliferation, as expected. Both the 24 hour and 8 hour exposures eventually ended with nearly equivalent (p=0.173 at 48 hours and p=0.603 at 72 hours) viabilities, however cells exposed to only 8 hours of liposomes showed much less cytotoxicity within the first 24 hours.

### Effect of Temperature at the Atomic Level

Based on the predicted equilibration ratios, the liposomes, which are near 100 nm, should have D:L ratios of 0.07 (e.g., as is illustrated in FIG. 12). The prediction assumes that the inner core of the liposome contains the same concentration of compound as the external environment. Since the encapsulation curtails at 1 hour (e.g., as is illustrated in FIG. 8B), the liposomes are assumed equilibrated by then, with D:L ratios at 54% of the maximum predicted. To understand the physics that underlie the equilibration, molecular dynamic simulations showed molecular interactions at the atomic level.

The membrane was composed of DSPC and cholesterol at a 2:1 ratio. Membranes were equilibrated for 1 ns at specified temperatures (37, 55, and 65° C.), and simulations of Doxorubicin diffusion through the membrane were conducted for 100 ns. In order to bypass the membrane, DXR molecules must first interact with the hydrophilic heads of the membrane. Once through this barrier, the molecule must also bypass the hydrophobic tail, which for a hydrophilic compound may be thermodynamically unfavorable.

The transition temperature of a lipid is a physical characteristic that dictates the order of the lipid membrane at varying temperatures, and is a crucial variable to consider in the engineering of liposomal delivery systems. Synthetic lipids are known to have a higher Tₘ than natural lipids. This is partially due to the increased purity of synthetic lipids, where natural lipids may exist with a range of tail lengths and saturation. DSPC is a synthetic, saturated, 18 carbon lipid with a Tₘ of 55° C. This elevated Tₘ imparts several favorable characteristics on the liposome, including enhanced in vivo stability and reduced clearance rates compared to natural lipids.

### CONCLUSION

Aspects, features, and benefits of the claimed disclosure(s) will become apparent from the information disclosed in the exhibits and the other applications as incorporated by reference. Variations and modifications to the disclosed systems and methods may be affected without departing from the spirit and scope of the novel concepts of the disclosure.

It will, nevertheless, be understood that no limitation of the scope of the disclosure is intended by the information disclosed in the exhibits or the applications incorporated by reference; any alterations and further modifications of the described or illustrated embodiments, and any further applications of the principles of the disclosure as illustrated therein are contemplated as would normally occur to one of general skill in the art to which the disclosure relates.

The foregoing description of the exemplary embodiments has been presented only for the purposes of illustration and description and is not intended to be exhaustive or to limit the disclosure to the precise forms disclosed. Many modifications and variations are possible in light of the above teaching.

While various aspects have been described in the context of a preferred embodiment, additional aspects, features, and methodologies of the claimed systems will be readily discernible from the description herein, by those of general skill in the art. Many embodiments and adaptations of the disclosure and claimed systems other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

The embodiments were chosen and described in order to explain the principles of the disclosures and their practical application so as to enable others of general skilled in the art to utilize the disclosures and various embodiments and with various modifications as are suited to the particular use contemplated. Alternative embodiments will become apparent to those of general skill in the art to which the present disclosures pertain without departing from their spirit and scope. Accordingly, the scope of the present disclosure is defined by the appended claims rather than the foregoing description and the exemplary embodiments described therein.

What is claimed is:

1. A method for making active agent-encapsulating small unilamellar liposomes comprising:
mixing an organic solvent and at least one lipid component to create a first solution;
heating the first solution to a first temperature within a predetermined range of a transition temperature of the at least one lipid component;
obtaining a second solution comprising:
an aqueous component; and
an active agent;
heating the second solution to the first temperature;
injecting the first solution into the second solution to form a mixture, wherein the at least one lipid component forms small unilamellar liposomes within the mixture;
incubating the mixture at the first temperature for a predetermined period of time, wherein:
incubating the mixture causes encapsulation of the active agent by the small unilamellar liposomes; and
the incubated mixture comprises active-agent encapsulating liposomes; and
concentrating the mixture via centrifugal filtration, wherein concentrating the mixture removes a substantial portion of non-encapsulated active agent from the mixture.

2. The method of claim 1, wherein the transition temperature is about 55 degrees Celsius and the predetermined range is between about 0-10 degrees Celsius.

3. The method of claim 1, wherein the first solution comprises the organic solvent at a concentration between about 5-10%.

4. The method of claim 3, wherein the organic solvent is isopropyl alcohol.

5. The method of claim 1, wherein injecting occurs at a rate of about 400 μL/min.

6. The method of claim 1, wherein injecting further comprises:
vortexing, at a predetermined rate, the second solution immediately before and throughout injecting the first solution into the second solution; and
vortexing, at the predetermined rate, the mixture that is formed after injecting the first solution.

7. The method of claim 6, wherein the predetermined rate is between about 400-800 revolutions per minute.

8. The method of claim 7, wherein mixing the first solution comprises vortexing the organic solvent at the predetermined rate immediately before, throughout and immediately after adding the at least one lipid component.

9. The method of claim 1, further comprising cooling the mixture to a second temperature that is below the transition temperature, wherein the cooled mixture comprises active agent-encapsulating small unilamellar liposomes.

10. The method of claim 9, wherein the second temperature is between about 0-5 degrees Celsius.

11. The method of claim 1, wherein the predetermined period of time is between about 1-2 hours.

12. The method of claim 1, further comprising concentrating the first solution via centrifugal filtration, wherein centrifugal filtration is performed at a rate of about 6000 g and repeated between about 5-10 times.

13. The method of claim 12, wherein centrifugal filtration is further performed using 100 kDa filtration tubes.

14. The method of claim 1, wherein the at least one lipid component is DSPC.

15. The method of claim 1, wherein the first solution further comprises cholesterol and the cholesterol also forms the small unilamellar liposomes.

16. A method for making active agent-encapsulating small unilamellar liposomes comprising:
mixing an isopropyl alcohol at a concentration between 5-10%, at least one lipid component and cholesterol to create a first solution, wherein the at least one lipid component and the cholesterol form small unilamellar liposomes within the first solution, wherein mixing further comprises:
vortexing the isopropyl alcohol, at a rate between about 400-800 revolutions per minute, immediately before and throughout adding the at least one lipid component and the cholesterol; and
vortexing the first solution at a rate between about 400-800 revolutions per minute;
heating the first solution to a first temperature between about 45-65 degrees Celsius;
concentrating the first solution by performing centrifugal filtration, wherein:
the centrifugal filtration is performed at a rate of about 6000 g;
the centrifugal filtration is performed using 100 kDa filtration tubes; and
the centrifugal filtration is repeated between about 5-10 times;
obtaining a second solution comprising:
an aqueous component; and
an active agent;
heating the second solution to the first temperature;
injecting the first solution into the second solution at a constant rate of about 400 μL/min to form a mixture, wherein injecting further comprises:
vortexing, at a predetermined rate between about 400-800 revolutions per minute, the second solution immediately before and throughout injecting the first solution into the second solution; and
vortexing, at the predetermined rate, the mixture that is formed after injecting the first solution;
incubating the mixture at the first temperature for about 1-2 hours, wherein incubating the mixture causes encapsulation of the active agent by the small unilamellar liposomes;
cooling the mixture to between about 0-5 degrees Celsius, wherein the cooled mixture comprises active agent-encapsulating small unilamellar liposomes; and
concentrating the mixture by performing centrifugal filtration, wherein concentrating the mixture removes a substantial portion of non-encapsulated active agent from the mixture, wherein:
the centrifugal filtration is performed at a rate of about 6000 g;
the centrifugal filtration is performed using 100 kDa filtration tubes; and
the centrifugal filtration is repeated between about 5-10 times.

17. A liposomal solution comprising small unilamellar liposomes, wherein the small unilamellar liposomes comprise:
a polydispersity of less than about 0.40;
a concentration, in the liposomal solution, between about 100 μM and 10 M; and
a mean diameter less than about 140 nm.

18. The liposomal solution of claim 17, wherein the small unilamellar liposomes further comprise an active agent that is encapsulated within the small unilamellar liposomes.

19. A method for making liposomes with low polydispersities, with low diameters, and with tunable potencies that does not comprise secondary processing, comprising:
mixing an alcohol solution of a charged lipid and an alcohol solution of cholesterol in a 2:1 molar ratio to form a mixed lipid solution comprising lipids, wherein the concentration of the lipids ranges between about 10M and about 100 μM; and
injecting, at a constant rate, the mixed lipid solution into an aqueous solution, heated to a temperature within 0-10 degrees of the phase transition temperature of the charged lipid molecule, to form a liposome solution having an alcohol concentration of between 5% to 10%, wherein the liposome solution is shaken or vortexed during and after the time of injecting.

20. The method of claim 19, wherein:
the alcohol of the alcohol solution is isopropyl alcohol;
the charged lipid is DSPC;
the transition temperature is 55° C.;
the constant rate is 400 μL/min;
the aqueous solution comprises at least one active agent that is encapsulated by liposomes formed in the aqueous solution; and
the liposomes comprise a polydispersity of less than about 0.40 and a mean diameter less than about 140 nm.
